SEQUENCE LISTING (85 sequences)

SEQ ID NO: 1 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFATIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 2 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 3 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 4 - protein, 584 aa, synthetic construct
GGGSTPLPLPQQQPPQQEPPPPPVPLASRAACEALKDGNGDMVWPNAAVVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 5 - protein, 584 aa, synthetic construct
GGGSTPLPLPQQQPPQQEPPPPPVPLASRAACEALKDGNGDMVWPNAATVVEVAAWRDPAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 6 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYAIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 7 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRLPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 8 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMQGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 9 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGGGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 10 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTDGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 11 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 12 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIGTDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 13 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLLMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 14 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDYGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 15 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNAYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 16 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAAKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 17 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKALVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 18 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAPDKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 19 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSNGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 20 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMASQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 21 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLAQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 22 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLVKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 23 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAAISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 24 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGIAGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 25 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWLTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 26 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTLQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 27 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLILKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 28 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLVSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 29 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLAQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 30 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVRAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 31 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWPWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 32 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSLMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 33 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDPAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 34 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADWYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 35 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTAANFACAAPP

SEQ ID NO: 36 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEASFACAAPP

SEQ ID NO: 37 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtaactttgcaaccattgcaaccgatggtggtcatgataatgccgttaatgataatccggatgcactgggcaccgttgcatttggtctggatccgcaggcacgtctggatatgggttataatagctatgatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 38 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagctatgatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 39 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcagtggttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 40 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 41 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatccggcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 42 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgcgatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 43 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtctgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 44 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatgcagggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 45 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtggcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 46 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccgatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 47 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtaactttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 48 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattagcaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 49 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctgctgatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 50 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggattatggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 51 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatgcggtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 52 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcagcgaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 53 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcactggttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 54 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcaccggataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 55 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcaacggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 56 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatggcgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 57 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctggcgcagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 58 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctggtgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 59 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcagcgattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 60 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattgcgggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 61 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggctgacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 62 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccctgcagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 63 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattctgaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 64 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctggtgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 65 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctggcgcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 66 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgagagcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 67 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttggccgtgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 68 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtctgatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 69 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacgcgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 70 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattggtatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccaattttgcatgtgcagcacctccgtaa

SEQ ID NO: 71 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgcggccaattttgcatgtgcagcacctccgtaa

SEQ ID NO: 72 - DNA, 1755 bp, synthetic construct
  source 1..1755: mol_type = other DNA
ggtggtggtagcacaccgctgccgctgcctcagcagcagcctccgcagcaagaaccgcctccgcctccggttccgctggcaagccgtgcagcatgtgaagcactgaaagatggtaatggtgatatggtttggccgaatgcagcaaccgttgttgaagttgcagcatggcgtgatgcagcaccggcaaccgcaagcgcagcagcactgccggaacattgtgaagttagcggtgcaattgccaaacgtaccggtattgatggttatccgtatgaaatcaaatttcgtctgcgtatgcctgcagaatggaatggtcgcttttttatggaaggtggtagcggcaccaatggtagcctgagcgcagccaccggtagcattggtggtggccagattgcaagcgcactgagccgtggttttgcagttattgcaaccgatggtggtcatgataatgccgttaatgataatccggatgcactgggcaccgttgcatttggtctggatccgcaggcacgtctggatatgggttataatagcgtggatcaggttacccaggcaggtaaagcagcagttgcacgtttttatggtcgtgcagcagataaaagctattttatcggttgtagcgaaggtggtcgtgaaggtatgatgctgagccagcgttttccgagccattatgatggtattgttgcgggtgcaccgggttatcagctgccgaaagcaggtattagtggtgcatggaccacacagagcctggcaccggcagccgttggcctggatgcacagggtgttccgctgattaacaaaagctttagtgatgccgatctgcatctgctgagtcaggcaattctgggcacctgtgatgccctggatggtctggcagatggcattgttgataactatcgtgcatgtcaggcagcatttgatccggcaacagcagcaaatccggcaaatggtcaggccctgcagtgtgttggtgcaaaaaccgcagattgtctgagtccggttcaggtgaccgcaattaaacgcgcaatggcaggtccggttaatagcgcaggtacaccgctgtataatcgttgggcatgggatgcaggtatgagcggtctgagcggtacaacctataatcaaggttggcgtagttggtggctgggtagctttaatagcagcgcaaataatgcccagcgtgttagcggttttagcgcacgtagctggctggttgattttgcaacccctccggaaccgatgccgatgacacaggttgcagcacgcatgatgaaattcgattttgatattgacccgctgaaaatttgggcaaccagcggtcagtttacccagagtagcatggattggcatggtgcaaccagcaccgatctggcagcctttcgtgatcgtggtggtaaaatgattctgtatcatggtatgtcagatgcagcctttagtgcactggatacagcagattattatgaacgtctgggtgcagcaatgcctggtgcagcaggttttgcacgtctgtttctggttccgggtatgaatcattgtagcggtggtccgggtacagatcgttttgatatgctgacccctctggttgcatgggttgaacgtggtgaagcaccggatcagattagcgcatggtctggtactccgggttattttggtgttgccgcacgtacccgtccgctgtgtccgtatccgcagatcgcacgttataaaggtagcggtgatattaacaccgaagccagctttgcatgtgcagcacctccgtaa

SEQ ID NO: 73 - protein, 584 aa, synthetic construct; identical to SEQ ID NO: 2

SEQ ID NO: 74 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADWYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 75 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDYGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADWYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 76 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFAVIATDGGHDNAVNDNPDALGTVAFGLDPQARLDYGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADWYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 77 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFAVIATDGGHDNAVNDNPDALGTVAFGLDPQARLDYGYNSYDQVTQAGKAAVARFYGRAADKSYFIGCSEGGREGMMLSQRFPSHYDGIVAGAPGFQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADWYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 78 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFAVIATDGGHDNAVNDNPDALGTVAFGLDPQARLDMGYNSYDQVTQAGKAAVARFYGRAADKSYFIGCSEGGREGMMLSQRFPSHYDGIVAGAPGFQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADWYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 79 - DNA, 1755 bp, synthetic construct
  source 1..1755: mol_type = other DNA
ggaggcggaagtactccgctccccctgccgcagcagcaaccaccacagcaggagccaccaccgcctcccgtgccattagcttcacgcgcagcttgtgaagcgctgaaagacggcaacggcgatatggtttggccgaacgcagcaacagtggttgaggttgccgcatggcgcgatgctgcgcctgctacggcctctgcagcagcgttacccgaacattgcgaagtgagtggtgccattgcgaaacgtacaggcatcgatggttatccatatgagatcaaattccgtctccgcatgcccgcagaatggaacggacgtttctttatggaaggaggttcaggtacaaacggaagtctgagtgccgctacaggtagtattggtggtgggcagattgccagcgctttgagtcgtgggtttgccgtgatagccaccgacggtggtcatgacaatgccgttaatgacaatcctgatgccttgggtactgtagcatttggactggacccgcaggcgcgtctggacatgggttacaactcctatgatcaggtcacacaggccgggaaggcggctgtggctcgcttttacggccgtgcggccgataaatcctattttattggctgtagtgaaggagggcgggaaggcatgatgctgtcacagcggtttccaagccactacgacggtattgtggcaggtgccccgggctaccagttacctaaagccgggatttctggggcgtggacaacacagtctcttgccccagccgccgtcggtctcgacgctcagggtgtcccgcttattaataaatcattcagcgatgccgacttacatctgctgagccaggcaattttaggtacatgtgatgcgctggatggtctggccgatggcatagtagacaattatagagcctgtcaagctgcctttgaccctgctaccgcagccaatccggctaacggccaagcactgcaatgtgttggcgcgaaaaccgcggattgtctgagtccagtgcaagtgactgcgataaaacgtgcaatggcgggcccggtcaatagtgctgggacacccttgtataatcgctgggcttgggacgcaggaatgtcgggattgagtggaaccacgtacaaccagggatggcgttcctggtggttgggctcgttcaacagcagcgctaataacgctcaacgagtgtcaggtttcagtgcgcgcagctggcttgtggattttgccacccccccggaaccaatgccgatgactcaggttgccgcgcgtatgatgaagtttgatttcgatattgatccgttgaaaatttgggccacctcaggacagtttacgcaaagttcgatggactggcacggggcgacatcaactgatctggcagcctttcgggacagagggggtaaaatgatcctttatcatggtatgtctgatgccgcattctctgcgctcgatacggccgactactacgaacgcttaggggctgctatgcctggtgcggctgggtttgcacgtctctttttggtgcctggtatgaatcactgttcaggaggccctggcaccgatagatttgatatgctcacccctctggttgcttgggtagagagaggtgaagctccggatcaaatcagtgcttggtctgggacccctggctacttcggtgtcgctgccagaacccgtccgctgtgtccatatccccagattgcgcgatataaagggtctggggatattaatacggaagctaattttgcgtgcgcggcgccgccgtaa

SEQ ID NO: 80 - DNA, 1755 bp, synthetic construct
  source 1..1755: mol_type = other DNA
ggcggtgggtcaacaccgttgccgttacctcaacagcagcccccgcaacaggaaccgccgccgccacctgttccgttagcatcacgtgcagcatgtgaggccctgaaagatggtaatggcgatatggtttggccaaacgccgctaccgttgtggaggtggctgcttggcgtgacgcggctcccgcaacagcgtctgcagccgccctgccagagcactgcgaagtttctggtgcaattgccaagcgtaccggaattgatggttatccttatgaaattaagttccgtctgcgcatgcccgctgaatggaatggacgttttttcatggagggtggctcgggtacaaatggttctctctccgctgcgacaggcagtatcggaggcggtcaaatcgcttcagcgttgtccagaggctttgctgttattgcaacggatggtggtcatgataacgcagtcaatgataaccctgacgcactgggaacggttgcgtttggtttggatcctcaggcacgtttagatatggggtacaattcttatgatcaggtaactcaggcagggaaagccgccgttgctcgtttttatggccgagctgctgacaagtcgtactttattgggtgcagcgaaggtgggcgagaaggcatgatgttgtcgcaacgatttcccagtcattacgacgggattgtggcgggtgcgccaggttaccagctgccgaaagcgggtatctcgggtgcttggacaacccagagtttagcgccggcggctgtaggcttagatgcacagggtgtcccattgattaataaatcattttctgatgctgacctccatctgttatcgcaggcgatcctcggaacctgcgatgcgctggatggcctggcagacggtatagtcgataattacagagcttgtcaagcggcgtttgaccctgcgaccgcggccaacccagcgaacgggcaggcacttcaatgtgttggtgcaaaaacagcggattgtctgtccccggtacaagtaacggcaatcaagcgtgcaatggctggtccggtgaattcggccggcacaccgctgtacaatcggtgggcgtgggacgcaggtatgtcaggtttatccggaacgacgtacaatcaagggtggagatcgtggtggctgggttcctttaactctagtgcaaacaacgcccagcgcgtctctggattttccgcgcgttcttggctggtcgatttcgcgacgccacccgaacccatgccgatgacccaggtagcggcacgcatgatgaagtttgattttgatatcgatcccctcaaaatttgggccactagtggccagtttacgcagtcaagtatggattggcatggcgccacgagtacagacttggcggcttttcgcgaccgcgggggtaagatgatcctgtaccacggcatgagcgatgctgcatttagtgccctcgatacagcagactggtatgaacgtcttggtgcagctatgccaggtgcagcaggctttgctcgcctgttcttggttccggggatgaaccattgcagcggtggtccgggtaccgaccgcttcgatatgctgacgccattagtagcttgggttgaacgtggggaagcccctgatcaaattagtgcatggtcgggcacacctgggtatttcggcgtggcggcccggacgagaccgctttgtccctatccccagattgcccggtataaaggtagtggtgacataaacaccgaagcgaattttgcgtgcgccgcgccgccctga

SEQ ID NO: 81 - DNA, 1755 bp, synthetic construct
  source 1..1755: mol_type = other DNA
ggcggtggctccacccctttacccttgccacagcagcaaccgccgcagcaagaacctccaccccctccggtccccctggcgtcgcgcgcagcgtgcgaagccttgaaagatggtaatggcgacatggtttggccaaacgccgccaccgtagttgaggtagcagcctggcgcgatgccgctccagccactgcctcggccgccgcgctcccggaacattgtgaagttagtggcgcaattgcgaaacgcacaggaattgacggctatccgtatgagatcaaattccggctgcgtatgccggcagaatggaacggccggttctttatggaaggcgggagtggaaccaatggatccctgtcagctgcgactggatccataggcggtgggcagatagcatcggctcttagccggggttttgcggtgatcgcgaccgatggcgggcatgacaacgcagtgaacgacaatcctgatgcgttgggcaccgttgcttttggtcttgatccgcaggctcggcttgattacgggtacaatagttatgaccaggtaacacaggctggtaaagcggcggtggcccgcttctatggcagagcagccgacaaatcgtatttcattggctgctctgaaggcggtcgcgaaggaatgatgctgtctcagcgatttccgtctcattatgatgggattgttgccggtgccccggggtatcaacttccgaaagcgggtatctccggtgcttggacaactcaatcgctggcccccgcggcggtgggtctggatgctcaaggcgtccctctcattaataagtcttttagtgatgctgatttgcatttgctgtcccaggccattctgggaacgtgcgatgctcttgacggtctcgccgatggcatagttgacaactatcgcgcctgtcaggccgcatttgacccagcgaccgcggctaatccagcgaatggtcaggcactgcaatgtgttggcgcaaaaacagccgactgcctgtcacctgttcaggtaacagcaattaaaagagcgatggctggcccagttaactcagcaggcaccccgctgtataatcgttgggcatgggatgccggtatgtcaggtttatccggaactacgtataatcagggatggcgttcttggtggctggggtcttttaactctagtgcaaacaatgcacaacgggtgagtggttttagtgcacggagctggcttgttgattttgcaacccctcctgagccgatgcctatgacacaagttgccgctcgcatgatgaaattcgatttcgacatagatccgctgaagatttgggcgacctcaggtcagtttacccagtcgtcaatggattggcacggcgcgacctcgactgacctcgccgcatttcgtgaccgtggaggcaaaatgattctgtatcacggtatgagcgatgctgccttttccgcgttggacacagcggattggtacgaacgtttgggcgctgcgatgccgggcgccgccgggttcgcccgattattcctcgtacctggaatgaatcactgttccggtgggccgggtacagaccgtttcgatatgctgaccccgcttgtagcctgggtcgagagaggcgaagccccggaccaaatatctgcgtggtcaggtactcctggttattttggtgtcgccgctcgtacacgtccattgtgtccgtaccctcagattgcacgttataaaggcagcggcgatattaacaccgaagctaatttcgcttgtgctgcaccaccctaa

SEQ ID NO: 82 - DNA, 1755 bp, synthetic construct
  source 1..1755: mol_type = other DNA
ggaggtggtagcacgcctttacctctgccgcagcagcagccgcctcaacaggaaccacccccgcctccggtgccgttagcatcccgtgcggcctgcgaagccctgaaggatggaaacggtgacatggtatggcccaacgcagctaccgtggtcgaagttgccgcttggcgcgacgctgcaccagccaccgcaagcgctgccgccctgcctgaacactgcgaagtgtccggtgccattgcaaaacgcacaggcatagatggctatccctatgagataaagtttcgcctgcgtatgcctgccgagtggaatgggcgtttttttatggaaggtggaagcggaaccaatggcagcctgagcgctgcaacaggttcaatcggcggtggtcaaattgcctccgccctttctcgcaattttgcagttattgctacggatggcggccacgataatgcagtcaacgataatccagacgctctggggaccgttgcctttggcctggatccgcaagcacgcttagactatggttataacagttacgatcaagtcacacaagcaggtaaagccgctgtcgcccgcttttatgggcgtgccgccgacaaatcttattttataggatgctctgaaggcggacgcgaaggtatgatgctgtcccaacgttttccgtcgcactatgatggaattgtcgctggtgcgcctggttatcaactgcccaaagccggcataagcggtgcttggacgacgcagtccctggcgccggcagcagttggattggatgctcaaggcgttccactgatcaataaaagcttcagcgatgcagacctgcacttactcagtcaggcgatcttgggaacttgcgacgcgctggatggtttggcagatggaattgtggataattacagagcttgtcaggcggcgtttgatccagctacagccgcaaacccggcgaatggacaggccctgcagtgcgtaggggccaaaacggcagattgtctgtccccggttcaagtaacagccattaagcgggccatggcaggtcccgttaattctgcaggtacgcccctgtacaaccggtgggcctgggatgcgggtatgtccggcctcagcggaacaacatataaccaaggttggcgatcttggtggctgggtagtttcaactcgagtgcaaataacgcacagagagtgtccggttttagtgctcggtcctggttggttgattttgccacaccaccagaacctatgcctatgacccaggtggctgcgcggatgatgaagtttgatttcgatattgacccccttaagatctgggcaacgagcggccagtttacacagagcagcatggattggcatggcgccacctctaccgatctggcggcttttcgtgaccgcggcggaaaaatgattttatatcacggcatgtcggatgcagccttcagcgcgcttgatactgcggattggtatgaacgcttaggcgcagctatgcctggagctgctggttttgccagactgttccttgtgccaggaatgaatcattgtagtggcggtcccggtactgatcgctttgacatgttaacgcctctggtggcctgggttgagcgtggtgaagcccctgatcaaatctcagcgtggtctggcacgccaggctatttcggtgttgcagctcgcacacgtcccctctgtccataccctcagatagcaagatacaaaggtagtggggacatcaataccgaggcaaacttcgcatgcgcggccccgccctga

SEQ ID NO: 83 - DNA, 1755 bp, synthetic construct
  source 1..1755: mol_type = other DNA
ggtggcggtagtaccccgctgccgctgccgcagcagcagccaccgcaacaggagccacctcctccgccggtccctttggcatcccgcgcagcttgtgaggctttgaaagacggtaatggagacatggtttggccgaatgctgcgactgtagtggaagttgcggcgtggcgtgatgcagcaccggcaactgcgagcgccgctgcgctccctgaacattgcgaggttagtggggctattgctaagcgtaccgggattgatggctatccatacgagattaaatttcgtctgcgcatgccagcggaatggaatggtagatttttcatggaaggtgggtccggcaccaatggctcgctcagcgctgcaacaggctccatcggcgggggtcagattgcgtcagccttatcgcgtaattttgctgttatcgctaccgatggcggccacgataatgcagttaacgataatccggacgctctggggactgttgcgtttggcttagatccgcaggccagactggattatgggtataattcgtacgatcaggtcacacaggcaggtaaagccgccgttgcccgcttttatgggcgggctgcggacaaatcatattttattggatgttcggaaggaggccgggaaggaatgatgttgagccaacgatttcctagccattatgacggcattgttgccggggcaccgggcttccaattgcctaaagcaggaatttcaggtgcgtggacgacccagtctctggcgcctgcggcagtaggactggacgcgcagggagtccctttgattaataaatccttctctgatgctgatttgcatcttttgtcccaagccattttaggcacttgcgatgcgctggacggcttagcagatggtatagttgataattatcgcgcctgccaggcggcttttgatcccgcaacagcagcgaaccctgcgaacggtcaggccctgcaatgtgtcggtgcaaaaaccgcggactgtttgtctccggtgcaagtgactgcaattaagcgtgcaatggctggaccggtgaactccgctggaacacccttgtacaaccgctgggcgtgggatgctggcatgagcggcctgtctgggacgacttataaccagggctggcgctcttggtggttaggttcatttaattcctccgcgaataatgcccagcgcgtatctggtttttctgcccgttcttggttggtggattttgctacgccgccagaaccgatgccgatgacgcaagtcgcggcacgaatgatgaagttcgactttgacatagacccgctgaaaatttgggcaacaagcggtcagtttacgcaaagcagcatggactggcacggagcaaccagcactgatctcgcggcgtttagagatcgtgggggaaaaatgattttatatcatggcatgtcagacgccgctttttccgctctggacacagccgactggtacgaacggctgggagcagctatgccaggggccgctggttttgcacgcttatttctggtgccaggtatgaaccattgtagtgggggccccggaaccgatcgctttgatatgcttaccccgcttgtagcgtgggttgaacgtggtgaagctcctgatcaaatttctgcctggagtggtacgcctgggtatttcggcgtggcggcgcgtactcgaccactgtgcccttaccctcaaattgcgcgttataaaggtagtggtgatattaacacagaggcgaactttgcctgtgctgccccgccctaa

SEQ ID NO: 84 - DNA, 1752 bp, synthetic construct
  source 1..1752: mol_type = other DNA
ggtggcagtacaccgcttccgcttccgcaacagcaaccgcctcaacaggaacctccgcctcctcccgtgcctctcgccagccgtgcagcctgcgaggcgcttaaagacgggaatggtgacatggtatggccaaacgcggccacggtggttgaggttgccgcgtggagagatgctgctccggcaacggcatccgccgctgcgttgccagaacactgtgaggtatcgggcgccatagccaaacgtaccggcattgatggctatccatacgaaattaaatttagactccgcatgccagcagagtggaacggacgtttttttatggagggtggttctggtacaaatggcagcttaagtgccgcgactggctccattggcggagggcagattgcatcagcgttgtctcgtaattttgctgttatcgccaccgacgggggacatgacaacgccgttaacgataaccctgacgccctgggcactgttgcctttggtcttgatccccaggcgcgtttagatatgggctataattcttacgatcaggttacacaggccggcaaggctgcagtagcacggttctacggtagagccgcagataaatcatattttataggttgctctgagggtgggcgtgaaggtatgatgctttctcagcgcttcccctcccattatgatggaatagtggctggtgctccaggcttccagctgccaaaagccggcatctcaggcgcatggaccacgcaaagcctcgctcctgcggcagttggtttggatgcgcagggcgtgccgttaatcaataagtctttctctgatgccgatttgcacctgctgagccaagcaattttaggaacttgcgatgccctggatgggttggcggacggtatagtcgataattatcgcgcatgtcaggccgccttcgatccggcaaccgcagctaatcccgccaatggccaggcgctgcagtgtgtaggtgcaaaaacagctgactgtctcagtcctgtccaggtaactgctattaagcgcgcgatggcaggtcctgtgaactcagcaggcacgccgttatataatcgctgggcctgggatgcggggatgtccgggctgtcaggtaccacttataatcagggttggagaagctggtggctgggatcatttaacagtagcgctaacaatgcacagcgcgtctctggatttagcgctcgctcctggttagtagattttgctactcccccagaaccgatgccgatgacgcaggttgctgctcgaatgatgaagtttgactttgatatcgaccctctgaagatttgggcgaccagtggtcagtttacacagtcctcaatggattggcatggcgctacgtctacggacttagcagcctttcgcgatcgcggtgggaaaatgatactgtatcacggcatgtcagatgcggcgtttagcgcgttggatacggctgattggtacgaacgtttaggtgcagctatgccgggtgccgcagggtttgcgcggctgtttttggttcccggtatgaatcactgttcgggaggtccgggtactgatcgctttgatatgcttaccccgcttgtagcctgggtggaacgtggagaggccccggatcagatctccgcctggagcggtaccccgggttattttggagtcgcggcacggactcgccctctgtgcccatatcctcagattgctagatataaaggtagcggcgacataaatacagaagccaactttgcatgcgcagcaccaccatga

SEQ ID NO: 85 - DNA, 5365 bp, synthetic construct
  source 1..5365: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgcgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccattgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

(19) United States
(12) Patent Application Publication
(10) Pub. No.: US 2024/0150739 A1
(43) Pub. Date: May 9, 2024

(54) ENZYME VARIANTS AND USES THEREOF

(71) Applicant: SAMSARA ECO PTY LIMITED, Waverton (CA)

(72) Inventors: Jake Wesley SAUNDERS, Waverton (AU); Matthew SPENCE, Waverton (AU); Vanessa VONGSOUTHI, Waverton (AU); Adam Michael DAMRY, Waverton (AU); Colin John JACKSON, Waverton (AU)

(21) Appl. No.: 18/507,504

(22) Filed: Nov. 13, 2023

Related U.S. Application Data
(63) Continuation of application No. PCT/AU2022/050455, filed on May 13, 2022.

(30) Foreign Application Priority Data
May 13, 2021 (AU) 2021901431

(51) Int. Cl. C12N 9/18; C08J 11/10; C12P 7/18; C12P 7/62

(52) U.S. Cl. CPC C12N 9/18; C08J 11/105; C12P 7/18; C12P 7/62; C08J 2367/02

(57) ABSTRACT

The present disclosure relates generally to a polypeptide having mono-(2-hydroxyethyl) terephthalate hydrolase (MHETase) activity, more specifically to a polypeptide comprising an amino acid sequence that (i) has at least 70% sequence identity to SEQ ID NO:1 and (ii) differs from SEQ ID NO:1 by an amino acid substitution at one or more positions selected from the group consisting of positions that correspond to amino acid positions 156 to 396, 398 to 410 and 425 to 603 of SEQ ID NO:1.

This application is a continuation of International Application No. PCT/AU2022/050455, filed May 13, 2022, which claims the benefit of priority to Australian Application No. 2021901431, filed May 13, 2021, the disclosures of which are incorporated by reference herein in their entirety, including any drawings.

This application contains a Sequence Listing, which is incorporated by reference in its entirety. The accompanying Sequence Listing text file, name, “2023-11-10 Sequence_Listing_ST26 042115-513C01US” was created on Nov. 10, 2023 and is 173 KB.

The present invention relates to novel enzymes, more particularly to recombinant enzymes that hydrolyse the ester bond of mono-(2-hydroxyethyl)terephthalic acid and uses thereof.

### BACKGROUND

All references, including any patent or patent application cited in this specification are hereby incorporated by reference to enable full understanding of the invention. Nevertheless, such references are not to be read as constituting an admission that any of these documents forms part of the common general knowledge in the art, in Australia or in any other country.

Global industrialization has had significant environmental impact, not least of which is an increase in the manufacture and reliance on plastic and plastic products. Whilst there is a growing effort to find suitable and environmentally sustainable alternatives to plastics, including their manufacture and disposal, such products remain a significant problem and contribute to a vast majority of environmental pollutants. One of the major contributors to this problem is polyethylene terephthalate (PET) and its waste products, millions of tons of which is produced globally every year. The environmental significance of this problem is attributed, at least in part, to the chemical nature of plastics, in particular PET based products, as they do not readily to decompose in nature.

Approaches to deal with the problem of plastic waste products have typically included incineration, disposal in landfill and mechanical disintegration. However, these approaches also have significant environmental impact. For instance, incineration of plastics produces potentially harmful byproducts that are released into the atmosphere; the decomposition rate of plastics in landfill is typically very slow and there is a risk that toxic materials will leach into groundwater; and mechanical disintegration is relatively expensive and there is often limited use for its byproducts.

More recently, biological (enzymatic) degradation of plastics has been considered as an alternative approach to reducing plastic waste accumulation. This approach includes the use of PETases, an esterase class of enzyme that catalyze the hydrolysis of PET to the monomeric mono-2-hydroxyethyl terephthalate (MHET). MHETase can also be utilized to hydrolyze the MHET to terephthalate and ethylene glycol. These hydrolysates can then be suitably recycled as material for the manufacture of new products, including plastics.

Whilst enzymatic degradation of plastics is an attractive alternative to alleviating the environmental impact of plastic waste products and their disposal, it has not yet seen widespread adoption, including because of its relative inefficiency, slow rate of enzymatic degradation and low levels of enzyme expression in common industrial host strains. Hence, there remains an urgent need for improved methods and reagents for the enzymatic degradation of plastics.

### SUMMARY OF THE INVENTION

In an aspect disclosed herein, there is provided is a polypeptide having mono-(2-hydroxyethyl) terephthalate hydrolase (MHETase) activity, wherein the polypeptide comprises an amino acid sequence that (i) has at least 70% sequence identity to SEQ ID NO:1 and (ii) differs from SEQ ID NO:1 by an amino acid substitution at one or more positions selected from the group consisting of positions that correspond to amino acid positions 156 to 396, 398 to 410 and 425 to 603 of SEQ ID NO:1.

In another aspect, there is provided a polypeptide comprises an amino acid sequence that differs from SEQ ID NO:1 by an amino acid substitution at one or more positions selected from the group consisting of:
(i) a position that corresponds to amino acid positions 156 of SEQ ID NO:1;
(ii) a position that corresponds to amino acid position 159 of SEQ ID NO:1;
(iii) a position that corresponds to amino acid position 192 of SEQ ID NO:1; and
(iv) a position that corresponds to amino acid position 503 of SEQ ID NO:1.

In an embodiment, the polypeptide differs from SEQ ID NO:1 by amino acid substitutions at positions that correspond to amino acid positions 159, 252 and 503 of SEQ ID NO:1. In another embodiment, the amino acid substitutions are T159V, Y252F and Y503W, or conservative amino acid substitutions of any of the foregoing.

In another embodiment, the polypeptide differs from SEQ ID NO:1 by amino acid substitutions at positions that correspond to amino acid positions 159, 192, 252 and 503 of SEQ ID NO:1. In another embodiment, the amino acid substitutions are T159V, M192Y, Y252F and Y503W, or conservative amino acid substitutions of any of the foregoing.

In another aspect, there is provided a polypeptide having mono-(2-hydroxyethyl) terephthalate hydrolase (MHETase) activity, wherein the polypeptide comprises an amino acid sequence that (a) has at least 70% sequence identity to SEQ ID NO:1, and (b) differs from SEQ ID NO:1 by an amino acid substitution at one or more positions selected from the group consisting of:
(i) a position that corresponds to amino acid positions 156 of SEQ ID NO:1;
(ii) a position that corresponds to amino acid position 159 of SEQ ID NO:1;
(iii) a position that corresponds to amino acid position 196 of SEQ ID NO:1;
(iv) a position that corresponds to amino acid position 197 of SEQ ID NO:1;
(v) a position that corresponds to amino acid position 260 of SEQ ID NO:1;
(vi) a position that corresponds to amino acid position 264 of SEQ ID NO:1;
(vii) a position that corresponds to amino acid position 267 of SEQ ID NO:1;
(viii) a position that corresponds to amino acid position 286 of SEQ ID NO:1; and
(ix) a position that corresponds to amino acid position 503 of SEQ ID NO:1.

In some embodiments, the polypeptide disclosed herein further comprises one or more of:
(i) increased recombinant expression in a host cell system;
(ii) increased whole cell activity; and
(iii) increased thermostability,
   when compared to an MHETase of SEQ ID NO:1.

The present disclosure also extends to a composition comprising the polypeptide as described herein.

The present disclosure also extends to a nucleic acid sequence encoding the polypeptide described herein. In an embodiment, the nucleic acid sequence encoding the polypeptide is selected from SEQ ID NOs: 37-72 and 79-84. In a preferred embodiment, the nucleic acid sequence encoding the polypeptide is selected from SEQ ID NOs:79-84.

The present disclosure also extends to an expression vector comprising the nucleic acid sequence described herein.

The present disclosure also extends to a host cell comprising the nucleic acid sequence or the expression vector described herein.

In another aspect, the present disclosure provides a method of producing a polypeptide having MHETase activity, the method comprising:
a) providing a nucleic acid sequence described herein;
b) expressing the nucleic acid sequence in a host cell culture, thereby producing the polypeptide; and
c) collecting the polypeptide produced in (b) from the host cell culture.

In yet another aspect, there is provided method of hydrolysing a mono-(2-hydroxyethyl) terephthalate, the method comprising exposing the mono-(2-hydroxyethyl) terephthalate to the polypeptide, the composition or the host cell described herein under conditions sufficient to convert the mono-(2-hydroxyethyl) terephthalate to terephthalate and ethylene glycol.

The present disclosure also extends to a method of degrading a plastic product comprising a polyester, the method comprising exposing the plastic product to the polypeptide, the composition or the host cell described herein.

In another embodiment, the methods disclosed herein comprise:
(i) exposing the PET to a polyethylene terephthalate esterase (PETase) under conditions sufficient for the PETase to catalyze the conversion of the PET to generate mono-(2-hydroxyethyl) terephthalate (MHET); and
(ii) simultaneously or sequentially, exposing the MHET generated in step (a) to the polypeptide, the composition or the host cell as described herein, under conditions sufficient for the polypeptide to catalyze the hydrolysis of the MHET to produce terephthalate and ethylene glycol.

The present disclosure also extends to a composition comprising the terephthalate and/or ethylene glycol recovered by the method disclosed herein.

In another aspect, there is provided a host cell genetically modified to express the polypeptide described herein.

### BRIEF DESCRIPTION OF THE DRAWINGS

FIG. 1 shows the amino acid sequences of the wild-type (WT) MHETase (SEQ ID NO:1) and the different consensus designs (SEQ ID NOs:2-36).

FIG. 2 shows the nucleic acid sequences of the wild-type (WT) MHETase (SEQ ID NO:37) and of the different consensus designs (SEQ ID NOs:38-72).

FIG. 3 shows the activity of MHETase variants (dA₄₆₅/dt (min⁻¹)) in whole cell suspension against an analogue of MHET (1-naphthyl terephthalate).

FIG. 4 shows the expression levels of wild type MHETase (WT; SEQ ID NO:1) and MHETase variants comprising point mutations, including the MHETase variant N156G+T159V, in soluble cell lysates by SDS-PAGE gel electrophoresis and staining with NTA-Atto550 (Sigma).

FIG. 5 shows the thermostability of purified wild type MHETase (WT), and MHETase variants comprising point mutations N156G+T159V, N156G+T159V+Y197V and N156G+T159V+YY503W, as determined by circular dichroism at 222 nm (Y-axis) and at temperatures ranging from 20-90° C. (X-axis).

FIG. 6 shows whole-cell suspension FastBlue assay results for all tested MHETase variants from each mutagenesis round. The bar height represents the average activity (dA₄₆₅/dt (min⁻¹)) measured for each variant (n≥2, individual measurements shown), and error bars represent the standard error mean of the measurements. Highlighted bars represent the variant used as parent in the following round of mutagenesis.

FIG. 7 shows SDS-PAGE gel of the selected MHETase variant from each round stained using ATTO550 and imaged under UV transillumination. The expected size of the MHETase variants (≈64 kDa) is indicated.

FIG. 8 shows size exclusion chromatogram of selected MHETase variants.

FIG. 9 shows a Michaelis-Menten plot for selected MHETase variants obtained using the chromogenic assay described herein. Each point represents the average initial rate of reaction from three technical replicates, each incubated with 6 nM MHETase and 4 mM Fast Blue B Salt. Error bars represent the standard error mean.

FIG. 10 shows thermostability of MHETase variants from three replicates measured by circular dichroism at 222 nm in Sodium Acetate pH 5.1. The data was fit to a two-state unfolding model (lines), with error bars corresponding to the standard error mean.

FIG. 11 shows HPLC assay comparing the activity of wild-type MHETase, Round 5 Y252F (R5), and reversions of R5 to the wild-type MHETase identity at positions 192, 156, 159, 252 and 503.

FIG. 12 shows whole-cell suspension FastBlue assay results for MHETase R5 reversion mutations. The mutations V159T, Y192M, F252Y, and W503Y were made in the background of MHETase R5 (MHETase 1252F of Round 5). The bar height represents the average activity measured for each variant (n≥2), and error bars represent the standard error mean.

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article, unless explicitly stated otherwise. By way of example, “an element” means one element or more than one element.

As used herein, the term “about” refers to a quantity, level, value, dimension, size, or amount that varies by as much as 10% (e.g, by 10%, 9%, 8%, 7%, 6%, 5%, 4%, 3%, 2% or 1%) to a reference quantity, level, value, dimension, size, or amount.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The present disclosure is predicated, at least in part, on the inventors' unexpected findings that certain modifications can be made to the amino acid sequence of a mono-(2-hydroxyethyl) terephthalate hydrolase (MHETase) that advantageously enhance its MHETase activity. Certain modifications also unexpectedly improved the expression of the modified MHETase when expressed in a host cell. Certain modifications surprisingly improved the whole cell activity of the modified MHETase when expressed in a host cell. Certain modifications also unexpectedly improved the thermostability of the enzyme. The present inventors have also unexpectedly found that substitutions can be made to amino acid residues that sit outside of the active site of wild type MHETase to enhance MHETase activity and expression when compared to the wild type MHETase. Thus, in an aspect disclosed herein, there is provided is a polypeptide having mono-(2-hydroxyethyl) terephthalate hydrolase (MHETase) activity, wherein the polypeptide comprises an amino acid sequence that (i) has at least 70% sequence identity to SEQ ID NO:1 and (ii) differs from SEQ ID NO:1 by an amino acid substitution at one or more positions that do not otherwise make contact with a polyester substrate of MHETase, and wherein the MHETase activity of the polypeptide is greater than the MHETase activity of the MHETase of SEQ ID NO:1.

In another aspect disclosed herein, there is provided is a polypeptide having mono-(2-hydroxyethyl) terephthalate hydrolase (MHETase) activity, wherein the polypeptide comprises an amino acid sequence that (i) has at least 70% sequence identity to SEQ ID NO:1 and (ii) differs from SEQ ID NO:1 by an amino acid substitution at one or more positions selected from the group consisting of positions that correspond to amino acid positions 156 to 396, 398 to 410 and 425 to 603 of SEQ ID NO:1.

By “at least 70%” is meant that the polypeptide shares at least 70%, preferably at least 70%, preferably at least 75%, preferably at least 80%, preferably at least 85%, preferably at least 90%, preferably at least 92%, preferably at least 94%, preferably at least 95%, preferably at least 96%, preferably at least 97%, preferably at least 98%, or more preferably 99% sequence identity to SEQ ID NO: 1. As the polypeptide described herein is a variant of the naturally-occurring (wild-type) MHETase of SEQ ID NO:1, it is to be understood that, in this context, “at least 70%” does not include 100% sequence identity across the entire sequence (residues 1-603 or residues 18-603) of SEQ ID NO:1. In some embodiments, the polypeptide may comprise amino acid insertions and/or deletions, such as at the N- and/or C-termini, as long as the modified polypeptide has greater MHETase activity when compared to the MHETase of SEQ ID NO:1, as described herein.

In another aspect, there is provided a polypeptide having mono-(2-hydroxyethyl) terephthalate hydrolase (MHETase) activity, wherein the polypeptide comprises an amino acid sequence that (a) has at least 70% sequence identity to SEQ ID NO:1, and (b) differs from SEQ ID NO:1 by an amino acid substitution at one or more positions selected from the group consisting of:
(i) a position that corresponds to amino acid positions 156 of SEQ ID NO:1;
(ii) a position that corresponds to amino acid position 159 of SEQ ID NO:1;
(iii) a position that corresponds to amino acid position 192 of SEQ ID NO:1; and
(iv) a position that corresponds to amino acid position 503 of SEQ ID NO:1.

In an embodiment, the amino acid sequence of the polypeptide differs from SEQ ID NO:11 by an amino acid substitution at a position that corresponds to amino acid position 156 of SEQ ID NO:1.

In an embodiment, the amino acid substitution at a position that corresponds to amino acid position 156 of SEQ ID NO:1 is N156G, or a conservative amino acid substitution thereof.

In an embodiment, the amino acid sequence of the polypeptide differs from SEQ ID NO:1 by an amino acid substitution at a position that corresponds to amino acid position 159 of SEQ ID NO:1.

In an embodiment, the amino acid substitution at a position that corresponds to amino acid position 159 of SEQ ID NO:1 is T159V, or a conservative amino acid substitution thereof.

In an embodiment, the amino acid sequence of the polypeptide differs from SEQ ID NO:1 by an amino acid substitution at a position that corresponds to amino acid position 252 of SEQ ID NO:1.

In an embodiment, the amino acid substitution at a position that corresponds to amino acid position 156 of SEQ ID NO:1 is Y252F, or a conservative amino acid substitution thereof.

In an embodiment, the amino acid sequence of the polypeptide differs from SEQ ID NO:1 by an amino acid substitution at a position that corresponds to amino acid position 503 of SEQ ID NO:1.

In an embodiment, the amino acid substitution at a position that corresponds to amino acid position 156 of SEQ ID NO:1 is Y503W, or a conservative amino acid substitution thereof.

In a particular embodiment, the amino acid sequence of the polypeptide differs from SEQ ID NO:1 by amino acid substitutions at positions that correspond to amino acid positions 159, 252 and 503 of SEQ ID NO:1. In a preferred embodiment, the amino acid substitutions are T159V, Y252F and Y503W, or conservative amino acid substitutions of any of the foregoing.

In a particular embodiment, the amino acid sequence of the polypeptide differs from SEQ ID NO:1 by amino acid substitutions at positions that correspond to amino acid positions 159, 192, 252 and 503 of SEQ ID NO:1. In a preferred embodiment, the amino acid substitutions are T159V, M192Y, Y252F and Y503W, or conservative amino acid substitutions of any of the foregoing.

In a particular embodiment, the amino acid sequence of the polypeptide differs from SEQ ID NO:1 by amino acid substitutions at positions that correspond to amino acid positions 159, 192 and 503 of SEQ ID NO:1. In a preferred embodiment, the amino acid substitutions are T159V, M192Y and Y503W, or conservative amino acid substitutions of any of the foregoing.

In a particular embodiment, the amino acid sequence of the polypeptide differs from SEQ ID NO:1 by amino acid substitutions at positions that correspond to amino acid positions 156, 159 and 503 of SEQ ID NO:1. In a preferred embodiment, the amino acid substitutions are N156G, T159V, and Y503W, or conservative amino acid substitutions of any of the foregoing.

In a particular embodiment, the amino acid sequence of the polypeptide differs from SEQ ID NO:1 by amino acid substitutions at positions that correspond to amino acid positions 156 and 159 and 503 of SEQ ID NO:1. In a preferred embodiment, the amino acid substitutions are N156G, T159V, and Y503W, or conservative amino acid substitutions of any of the foregoing.

In another aspect, there is provided a polypeptide having mono-(2-hydroxyethyl) terephthalate hydrolase (MHETase) activity, wherein the polypeptide comprises an amino acid sequence that (a) has at least 70% sequence identity to SEQ ID NO:1, and (b) differs from SEQ ID NO:1 by an amino acid substitution at one or more positions selected from the group consisting of:
(i) a position that corresponds to amino acid positions 156 of SEQ ID NO:1;
(ii) a position that corresponds to amino acid position 159 of SEQ ID NO:1;
(iii) a position that corresponds to amino acid position 196 of SEQ ID NO:1;
(iv) a position that corresponds to amino acid position 197 of SEQ ID NO:1;
(v) a position that corresponds to amino acid position 260 of SEQ ID NO:1;
(vi) a position that corresponds to amino acid position 264 of SEQ ID NO:1;
(vii) a position that corresponds to amino acid position 267 of SEQ ID NO:1;
(viii) a position that corresponds to amino acid position 286 of SEQ ID NO:1; and
(ix) a position that corresponds to amino acid position 503 of SEQ ID NO:1,
   wherein the polypeptide has greater whole cell MHETase activity when compared to a polypeptide of SEQ ID NO:1.

In an embodiment, the amino acid sequence of the polypeptide differs from SEQ ID NO:1 by an amino acid substitution at a position that corresponds to amino acid position 156 of SEQ ID NO:1.

In an embodiment, the amino acid substitution at a position that corresponds to amino acid position 156 of SEQ ID NO:1 is N156G, or a conservative amino acid substitution thereof.

In an embodiment, the amino acid sequence of the polypeptide differs from SEQ ID NO:1 by an amino acid substitution at a position that corresponds to amino acid position 159 of SEQ ID NO:1.

In an embodiment, the amino acid substitution at a position that corresponds to amino acid position 159 of SEQ ID NO:1 is T159V, or a conservative amino acid substitution thereof.

In an embodiment, the amino acid sequence of the polypeptide differs from SEQ ID NO:1 by an amino acid substitution at a position that corresponds to amino acid position 196 of SEQ ID NO:1.

In an embodiment, the amino acid substitution at a position that corresponds to amino acid position 196 of SEQ ID NO:1 is S196A, or a conservative amino acid substitution thereof.

In an embodiment, the amino acid sequence of the polypeptide differs from SEQ ID NO:1 by an amino acid substitution at a position that corresponds to amino acid position 197 of SEQ ID NO:1.

In an embodiment, the amino acid substitution at a position that corresponds to amino acid position 197 of SEQ ID NO:1 is Y197V, or a conservative amino acid substitution thereof.

In an embodiment, the amino acid sequence of the polypeptide differs from SEQ ID NO:1 by an amino acid substitution at a position that corresponds to amino acid position 260 of SEQ ID NO:1.

In an embodiment, the amino acid substitution at a position that corresponds to amino acid position 260 of SEQ ID NO:1 is S260A, or a conservative amino acid substitution thereof.

In an embodiment, the amino acid sequence of the polypeptide differs from SEQ ID NO:1 by an amino acid substitution at a position that corresponds to amino acid position 264 of SEQ ID NO:1.

In an embodiment, the amino acid substitution at a position that corresponds to amino acid position 264 of SEQ ID NO:1 is S264L, or a conservative amino acid substitution thereof.

In an embodiment, the amino acid sequence of the polypeptide differs from SEQ ID NO:1 by an amino acid substitution at a position that corresponds to amino acid position 267 of SEQ ID NO:1.

In an embodiment, the amino acid substitution at a position that corresponds to amino acid position 267 of SEQ ID NO:1 is S267A, or a conservative amino acid substitution thereof.

In an embodiment, the amino acid sequence of the polypeptide differs from SEQ ID NO:1 by an amino acid substitution at a position that corresponds to amino acid position 286 of SEQ ID NO:1.

In an embodiment, the amino acid substitution at a position that corresponds to amino acid position 286 of SEQ ID NO:1 is S286A, or a conservative amino acid substitution thereof.

In an embodiment, the amino acid sequence of the polypeptide differs from SEQ ID NO:1 by an amino acid substitution at a position that corresponds to amino acid position 503 of SEQ ID NO:1.

In an embodiment, the amino acid substitution at a position that corresponds to amino acid position 503 of SEQ ID NO:1 is Y503W, or a conservative amino acid substitution thereof.

The present disclosure also contemplates combinations of amino acid substitutions at two or more (e.g., 2, 3, 4, 5, 6, 7, 8, 9, 10 and so on) positions corresponding to positions in SEQ ID NO:1, as described herein. In an embodiment, the polypeptide comprises a combination of amino acid substitutions at at least 2, preferably at at least 3, preferably at at least 4, preferably at at least 5, preferably at at least 6, preferably at at least 7, preferably at at least 8, preferably at at least 9, or more preferably at at least 10 positions corresponding to positions in SEQ ID NO:1, as described herein.

In an embodiment, the amino acid sequence of the polypeptide differs from SEQ ID NO:1 by amino acid substitutions at positions that correspond to amino acid positions 156, 159 and 197 of SEQ ID NO:1.

In an embodiment, the amino acid substitutions are N156G, T159V and Y197V, or conservative amino acid substitutions of any of the foregoing.

The present disclosure also extends to a composition comprising the polypeptide as described herein.

The present disclosure also extends to a nucleic acid sequence encoding the polypeptide described herein.

The present disclosure also extends to an expression vector comprising the nucleic acid sequence described herein.

The present disclosure also extends to a host cell comprising the nucleic acid sequence or the expression vector described herein.

In another aspect, the present disclosure provides a method of producing a polypeptide having MHETase activity, the method comprising:
a) providing a nucleic acid sequence described herein;
b) expressing the nucleic acid sequence in a host cell culture, thereby producing the polypeptide; and
c) collecting the polypeptide produced in (b) from the host cell culture.

In yet another aspect, there is provided method of hydrolysing a mono-(2-hydroxyethyl) terephthalate, the method comprising exposing the mono-(2-hydroxyethyl) terephthalate to the polypeptide, the composition or the host cell described herein under conditions sufficient to convert the mono-(2-hydroxyethyl) terephthalate to terephthalate and ethylene glycol.

The present disclosure also extends to a method of degrading a plastic product comprising a polyester, the method comprising exposing the plastic product to the polypeptide, the composition or the host cell described herein.

In an embodiment, the polyester is selected from the group consisting of polylactic acid (PLA), polytrimethylene terephthalate (PTT), polybutylene terephthalate (PBT), polyethylene isosorbide terephthalate (PEIT), polyethylene terephthalate (PET) polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate) (PEA), poly(glycolic acid) (PGA), poly(lactic-co-glycolic acid) (PLGA) and combinations of any of the foregoing. In an embodiment the polyester is polyethylene terephthalate (PET).

In another embodiment, the methods disclosed herein comprise:
a) exposing the PET to a polyethylene terephthalate esterase (PETase) under conditions sufficient for the PETase to catalyze the conversion of the PET to generate mono-(2-hydroxyethyl) terephthalate (MHET); and
b) simultaneously or sequentially, exposing the MHET generated in step (a) to the polypeptide of any one of claims 1 to 21, the composition of claim 22 or the host cell of claim 25, under conditions sufficient for the polypeptide to catalyze the hydrolysis of the MHET to produce terephthalate and ethylene glycol.

In an embodiment, the method further comprises recovering the terephthalate and/or ethylene glycol produced in step (b).

The present disclosure also extends to a composition comprising the terephthalate and/or ethylene glycol recovered by the method disclosed herein.

In another aspect, there is provided a host cell genetically modified to express the polypeptide described herein.

Herein, the terms “peptide”, “polypeptide”, “protein”, “enzyme” is to be understood as referring to a chain of amino acids linked by peptide bonds, irrespective of the number of amino acids forming said chain. The amino acids are typically represented by their one-letter or three-letters code, according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr).

The term “hydrolase” refers to an enzyme which belongs to a class of hydrolases classified as EC 3 according to Enzyme Nomenclature that catalyzes the hydrolysis of peptide bonds in a peptide or a protein in order to produce shorter peptides.

The terms “wild-type” or “parent” are used interchangeably herein to denote a naturally-occurring isoform of a polypeptide; that is, as it appears in nature. In the present disclosure, the wild-type polypeptide refers to the mono-(2-hydroxyethyl) terephthalic acid hydrolase having the amino acid sequence as set forth in SEQ ID NO: 1 (EC 3.1.1.102; UniProt Accession No. A0A0K8P8E7).

As noted by Palm et al. (2019, Nat. Comms. 10:1717), two recently discovered bacterial enzymes that specifically degrade polyethylene terephthalate (PET) represent a promising solution to an otherwise environmentally burdensome polyester containing product. First, Ideonella sakaiensis PETase, a structurally well-characterized α/β-hydrolase fold enzyme, converts PET to mono-(2-hydroxyethyl) terephthalate (MHET). MHETase, the second key enzyme, hydrolyzes MHET to the PET educts terephthalate and ethylene glycol (Palm et al. (2019, Nat. Comm., 10:1717), Sagong et al. (2020, ACS Catal. 10:4805) and Yoshida et al. (2020, Science, 352(6278):1196).

The amino acid and nucleic acid sequences of wild-type MHETases will be familiar to persons skilled, illustrative examples of which include SEQ ID NO:1.

The terms “mutant” and “variant” may be used interchangeably herein to refer to a polypeptide comprising an amino acid sequence that is derived from SEQ ID NO:1 and further comprising a modification or alteration (e.g., a substitution, insertion, and/or deletion), at one or more (e.g., several) positions and having enhanced MHETase activity when compared to the polypeptide of SEQ ID NO:1. Such variants may be obtained by various techniques well known in the art, illustrative examples of which include site-directed mutagenesis, random mutagenesis and synthetic oligonucleotide construction. The terms “modification”, “alteration”, “substitution” and the like, as used herein in relation to an amino acid residue or position, typically mean that the amino acid in the particular position has been modified compared to the amino acid of the wild-type or parent polypeptide.

Suitable substitutions may include the replacement of an amino acid residue by another selected from the naturally-occurring standard 20 amino acid residues, rare naturally occurring amino acid residues (e.g., hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, omithine, norleucine, norvaline), and non-naturally occurring amino acid residue, often made synthetically, (e.g., cyclohexyl-alanine). Preferably, the substitution comprises the replacement of an amino acid residue by another selected from the naturally-occurring standard 20 amino acid residues (G, P, A, V, L, I, M, C, F, Y, W, H, K, R, Q, N, E, D, S and T). The modification or alteration may be identified herein using the following terminology: Y197V denotes that amino acid residue Tyrosine (Y) at position 197 of the parent polypeptide sequence is substituted for a Valine (V). Y197V/I/M denotes that amino acid residue Tyrosine (Y) at position 197 of the parent sequence may be substituted for one of the following amino acids: Valine (V), Isoleucine (I), or Methionine (M). The substitution can be a conservative or non-conservative substitution. Examples of conservative substitutions will be familiar to persons skilled in the art, illustrative examples of which include substitutions within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

Unless otherwise specified, where the positions disclosed in the present application are numbered by reference to the amino acid sequence set forth in SEQ ID NO:1, the numbering of those positions corresponds to the same positions of the wild-type sequence as it appears in UniProt Accession No. A0A0K8P8E7 (which comprises SEQ ID NO:1 and a 19 amino acid N-terminal signal sequence). In this context, the term “corresponding to”, when used in reference to an amino acid position, is intended to mean an amino acid position in a polypeptide sequence when that position is aligned with the equivalent or corresponding position in the sequence set forth in A0A0K8P8E7.

As used herein, the term “sequence identity” or “identity” refers to the number (or fraction expressed as a percentage %) of matches (identical amino acid residues) between two polypeptide sequences. In a preferred embodiment, the sequence identity is determined by comparing the sequences when aligned so as to maximize overlap and identity while minimizing sequence gaps. Sequence identity may be determined using any of a number of mathematical global or local alignment algorithms known to persons skilled in the art, depending on the length of the two sequences. Sequences of similar lengths may be aligned using a global alignment algorithms (e.g., Needleman and Wunsch algorithm; Needleman and Wunsch, 1970), which aligns the sequences optimally over the entire length, while sequences of substantially different lengths are preferably aligned using a local alignment algorithm (e.g., Smith and Waterman algorithm (Smith and Waterman, 1981) or Altschul algorithm (Altschul et al., 1997; Altschul et al., 2005)). Alignment for the purposes of determining percent amino acid sequence identity can be achieved by any means available to persons skilled in the art, illustrative examples of which include publicly available computer software, such as is available at http://blast.ncbi.nlm.nih.gov/ or imp://www.ebi.ac.uk/Tools/emboss/). Persons skilled in the art can readily determine appropriate parameters for measuring alignment, including any algorithms needed to achieve maximal alignment over the full length of the sequences being compared. As used herein, % sequence identity typically refers to values generated using pair wise sequence alignment that creates an optimal global alignment of two sequences (e.g., using the Needleman-Wunsch algorithm), where all search parameters are set to default values, e.g., Scoring matrix=BLOSUM62, Gap open=10, Gap extend=0.5, End gap penalty=false, End gap open=10 and End gap extend=0.5.

The term “recombinant”, as used herein, typically refers to a nucleic acid construct, a vector, a polypeptide or a cell produced by genetic engineering.

The term “expression”, as used herein, typically refers to any step involved in the production of a polypeptide, such as by transcription, post-transcriptional modification, translation, post-translational modification, and secretion.

The term “expression cassette” denotes a nucleic acid construct comprising a coding region, and suitably a regulatory region to which the coding region is operably linked.

The term “expression vector” typically means a DNA or RNA molecule that comprises an expression cassette. The expression vector may be a linear or circular double stranded DNA molecule.

The term “polymer”, as used herein, typically refers to a chemical compound or a mixture of compounds whose structure is made up of multiple monomers (repeat units) linked by covalent chemical bonds. Within the context of the invention, the term polymer includes natural or synthetic polymers, constituted of a single type of repeat unit (i.e., homopolymers) or of a mixture of different repeat units (i.e., copolymers or heteropolymers).

As used herein, the terms “polyester containing material”, “polyester containing product” and the like are to be understood as refers to a product, such as plastic product, comprising at least one polyester in crystalline, semi-crystalline or totally amorphous form. The polyester containing material may refer to any item made from at least one plastic material, such as plastic sheet, tube, rod, profile, shape, film, massive block, fiber, textiles, etc., which contains at least one polyester, and possibly other substances or additives, such as plasticizers, mineral or organic fillers. In an embodiment, the polyester containing material is a textile or fabric comprising at least one polyester containing fiber. In another embodiment, the polyester containing material is a plastic compound, or plastic formulation, in a molten or solid state, suitable for making a plastic product.

Suitable polyesters will be familiar to persons skilled in the art, illustrative examples of which include polylactic acid (PLA), polyethylene terephthalate (PET), polytrimethylene terephthalate (PTT), polybutylene terephthalate (PBT), polyethylene isosorbide terephthalate (PEIT), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), and poly(ethylene adipate) (PEA). Thus, in an embodiment, the polyester is selected from the group consisting of polylactic acid (PLA), polyethylene terephthalate (PET), polytrimethylene terephthalate (PTT), polybutylene terephthalate (PBT), polyethylene isosorbide terephthalate (PEIT), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA) and combinations of any of the foregoing.

As noted elsewhere herein, the present inventors have developed variants of a naturally-occurring MHETase of SEQ ID NO:1 that exhibiting increased MHETase activity in comparison to the parent polypeptide of SEQ ID NO:1. The MHETase variants also display enhanced expression in a recombinant host cell system. More particularly, the inventors have developed novel MHETase having superior properties for use in industrial processes. With the aim improving the activity of hydrolases, in particular MHETase, in conditions at which industrial production of degradable plastic products can be performed and/or environmental degradation of plastic products can be obtained, the inventors have developed novel MHETase derived from the wild-type MHETase of SEQ ID NO:1 that unexpectedly show higher MHETase activity when compared to this parent hydrolase. The MHETase variants disclosed herein are particularly suited for the degradation of plastic products, in particular those containing PET. Moreover, the inventors have surprisingly found that amino acid residues that are not otherwise intended to contact a polyester substrate in the structure of the protein may be advantageously modified to enhance MHETase activity. Thus, in another aspect disclosed herein, there is provided is a polypeptide having mono-(2-hydroxyethyl) terephthalate hydrolase (MHETase) activity, wherein the polypeptide comprises an amino acid sequence that (i) has at least 70% sequence identity to SEQ ID NO:1 and (ii) differs from SEQ ID NO:1 by an amino acid substitution at one or more positions that do not make contact with a polyester substrate of the MHETase, and wherein the MHETase activity of the polypeptide is greater than the MHETase activity of the MHETase of SEQ ID NO:1. The term “contact”, in this context, typically refers to direct contact made by amino acid residues of the MHETase of SEQ ID NO:1 with a polyester substrate thereof. Amino acid residues of SEQ ID NO:1 that make contact with a polyester substrate thereof will be familiar to persons skilled in the art. Those residues are also described in Sagong et al. (2020, ACS Catal. 10:4805) and include R411, S416 and F424 of SEQ ID NO:1. In an embodiment, the polypeptide comprises an amino acid sequence that differs from SEQ ID NO:1 by an amino acid substitution at one or more positions that are outside of the active site of the MHETase of SEQ ID NO:1. The term “active site” typically refers to the region of SEQ ID NO:1 that is capable of making contact with and hydrolyzing the polyester substrate (i.e., MHET). Amino acid positions of SEQ ID NO:1 that lie outside of the active site of the MHETase of SEQ ID NO:1 will be familiar to persons skilled in the art.

In the context of the present disclosure, reference to increased or enhanced MHETase activity may include one or more of the following: increased ability of the polypeptide to hydrolyze MHET, when compared to the MHETase of SEQ ID NO:1; increased recombinant expression in a host cell system when compared to MHETase of SEQ ID NO:1; increased whole cell activity when compared to MHETase of SEQ ID NO:1; and increased thermostability when compared to MHETase of SEQ ID NO:1.

In an embodiment, the polypeptide as disclosed herein comprises increased recombinant expression of the MHETase in a host cell system, when compared to MHETase of SEQ ID NO:1.

In another embodiment, the polypeptide as disclosed herein comprises increased thermostability when compared to MHETase of SEQ ID NO:1.

In another embodiment, the polypeptide as disclosed herein comprises increased whole cell activity when compared to MHETase of SEQ ID NO:1. As used herein, the term whole cell activity typically refers to the ability of the MHETase to hydrolyze MHET when expressed in a host cell system.

In a further embodiment, the polypeptide as disclosed herein comprises increased thermostability and increased whole cell activity when compared to MHETase of SEQ ID NO:1.

In a further embodiment, the polypeptide as disclosed herein comprises increased recombinant expression of the MHETase in a host cell system, increased thermostability and increased whole cell activity when compared to MHETase of SEQ ID NO:1.

In an embodiment, the MHETase activity of the polypeptide described herein is similar to the MHETase activity of the MHETase of SEQ ID NO:1. In an embodiment, the MHETase activity of the polypeptide described herein is increased by at least about 5%, preferably by at least about 10%, preferably by at least about 20%, preferably by at least about 30%, preferably by at least about 40%, preferably by at least about 50%, preferably by at least about 100%, preferably by at least about 200%, preferably by at least about 300%, preferably by at least about 400%, preferably by at least about 500%, preferably by at least about 600%, preferably by at least about 700%, preferably by at least about 800%, preferably by at least about 900%, or more preferably by at least about 1,000% or more in comparison to the MHETase of SEQ ID NO:1. Suitable methods of determining or measuring MHETase activity of a polypeptide will be familiar to persons skilled in the art, an illustrative example of which is described elsewhere herein. Other illustrative examples are described in Palm et al. (2019, Nat. Comm., 10:1717), Sagong et al. (2020, ACS Catal. 10:4805) and Yoshida et al. (2020, Science, 352(6278):1196), the contents of which are incorporated herein by reference in their entirety. In an embodiment, the MHETase activity is increased by at least about 5%, preferably by at least about 10%, preferably by at least about 20%, preferably by at least about 30%, preferably by at least about 40%, preferably by at least about 50%, preferably by at least about 100%, preferably by at least about 200%, preferably by at least about 300%, preferably by at least about 400%, preferably by at least about 500%, preferably by at least about 600%, preferably by at least about 700%, preferably by at least about 800%, preferably by at least about 900%, or more preferably by at least about 1,000% or more in comparison to the MHETase of SEQ ID NO:1 when determined by a chromogenic assay using 1-Naphthyl Terephthalate (1NT) as a substrate.

MHETase activity may be assigned an absolute value or a value relative to the MHETase activity of a comparator (e.g., the MHETase of SEQ ID NO:1). In an embodiment, the MHETase activity is measured as the rate of monomers and/or oligomers (e.g., in mg) released per hour and per mg of enzyme under suitable conditions of temperature, pH and buffer.

The MHETase activity can be measured or assayed using a purified enzyme. Alternatively, MHETase activity can be measured as a function of the activity of the enzyme when recombinantly expressed in a host cell system (also referred to herein as cellular catalytic activity or whole cell activity).

Advantageously, the polypeptide described herein exhibits increased or enhanced recombinant expression in a host cell by at least about 5%, preferably by at least about 10%, preferably by at least about 20%, preferably by at least about 30%, preferably by at least about 40%, preferably by at least about 50%, preferably by at least about 100%, preferably by at least about 200%, preferably by at least about 300%, preferably by at least about 400%, preferably by at least about 500%, preferably by at least about 600%, preferably by at least about 700%, preferably by at least about 800%, preferably by at least about 900%, or more preferably by at least about 1,000% in comparison to the MHETase of SEQ ID NO:1.

Advantageously, the polypeptide described herein exhibits an MHETase activity at least in a range of temperatures from about 10° C. to about 60° C., preferably from about 20° C. to about 680° C., preferably from about 30° C. to about 60° C., more preferably from about 40° C. to about 60° C., even more preferably from about 40° C. to about 50° C., even more preferably at about 45° C. In an embodiment, the polypeptide described herein exhibits MHETase activity at a temperature from about 10° C. to about 60° C., preferably from about 20° C. to about 60° C., preferably from about 30° C. to about 60° C., more preferably from about 40° C. to about 60° C., even more preferably from about 40° C. to about 50° C., or even more preferably at about 45° C. In an embodiment, the MHETase activity is measurable at a temperature between about 40° C. and about 60° C., preferably between about 40° C. and about 50° C., or even more preferably at about 45° C. In another particular embodiment, the polyester degrading activity is still measurable at a temperature between about 10° C. and about 30° C., preferably between about 15° C. and about 28° C., corresponding to the mean temperature in the natural environment (ambient temperature).

In an embodiment, the polypeptide comprises MHETase activity at a temperature from about 10° C. to about 60° C., preferably from about 20° C. to about 60° C., preferably from about 30° C. to about 60° C., more preferably from about 40° C. to about 60° C., even more preferably from about 40° C. to about 50° C., or even more preferably at about 45° C. of at least about 5%, preferably by at least about 10%, preferably by at least about 20%, preferably by at least about 30%, preferably by at least about 40%, preferably by at least about 50%, preferably by at least about 100%, preferably by at least about 200%, preferably by at least about 300%, preferably by at least about 400%, preferably by at least about 500%, preferably by at least about 600%, preferably by at least about 700%, preferably by at least about 800%, preferably by at least about 900%, or more preferably by at least about 1,000% or more in comparison to the MHETase activity of SEQ ID NO:1 at the same temperature.

In another particular embodiment, the polypeptide described herein has increased MHETase activity, when compared to the polypeptide of SEQ ID NO:1, at a temperature of between about 10° C. and about 60° C., preferably between about 20° C. and about 60° C., preferably from about 30° C. to about 60° C., preferably between about 40° C. and about 60° C., preferably between about 40° C. and about 50 oC, or more preferably at about 45° C. In an embodiment, the polypeptide described herein has MHETase activity at between about 20 oC to about 6045° C. of at least about 5%, preferably by at least about 10%, preferably by at least about 20%, preferably by at least about 30%, preferably by at least about 40%, preferably by at least about 50%, preferably by at least about 100%, preferably by at least about 200%, preferably by at least about 300%, preferably by at least about 400%, preferably by at least about 500%, preferably by at least about 600%, preferably by at least about 700%, preferably by at least about 800%, preferably by at least about 900%, or more preferably by at least about 1,000% or more in comparison to the MHETase activity of SEQ ID NO:1 at the same temperature.

In another embodiment, the polypeptide described herein has increased MHETase activity, compared to the polypeptide of SEQ ID NO:1, at a temperature between about 10° C. and about 30° C., preferably between about 15° C. and about 30° C., even more preferably between about 20° C. and about 30° C., or even more preferably at about 28° C. In an embodiment, the polypeptide described herein has MHETase activity at a temperature between about 10° C. and about 30° C. of at least about 5%, preferably by at least about 10%, preferably by at least about 20%, preferably by at least about 30%, preferably by at least about 40%, preferably by at least about 50%, preferably by at least about 100%, preferably by at least about 200%, preferably by at least about 300%, preferably by at least about 400%, preferably by at least about 500%, preferably by at least about 600%, preferably by at least about 700%, preferably by at least about 800%, preferably by at least about 900%, or more preferably by at least about 1,000% or more in comparison to the MHETase activity of SEQ ID NO:1 at the same temperature.

In an embodiment, the polypeptide described herein exhibits a measurable MHETase activity at least in a range of pH from 5 to 11, preferably in a range of pH from 6 to 10, more preferably in a range of pH from 6.5 to 9, even more preferably in a range of pH from 7 to 8.

Advantageously, the thermostability of the polypeptide described herein is not significantly impaired compared to the polypeptide of SEQ ID NO:1. In some embodiments, the thermostability of the polypeptide described herein is improved when compared to the thermostability of the polypeptide of SEQ ID NO:1. The term “improved thermostability” or “increased thermostability”, as used herein, indicates an increased ability of the enzyme to resist changes in its chemical and/or physical structure at higher temperatures, more specifically at temperature between 40° C. and 60° C., as compared to the polypeptide of SEQ ID NO:1. In an embodiment, the polypeptides described herein have an increased half-life at a temperature between 40° C. and 60° C., as compared to the polypeptide of SEQ ID NO:1. The polypeptides described herein may exhibit a higher or equivalent melting temperature (Tm) as compared to the polypeptide of SEQ ID NO:1. In some embodiments, the polypeptide described herein shows improved thermostability at a temperature of between 40° C. and 68° C. as compared to the polypeptide of SEQ ID NO:1.

The thermostability of a polypeptide may be evaluated by any suitable means known to persons skilled in the art. For example, thermostability can be assessed by measuring the residual MHETase activity of the polypeptide after incubation at different temperatures. The ability to perform multiple rounds of MHETase-mediated hydrolysis at different temperatures can also be evaluated. Differential Scanning Fluorimetry (DSF) may also be used to assess the thermostability of the polypeptide. Circular dichroism may also be used to measure thermostability of the polypeptides described herein, including their melting temperatures (Tm). The term “melting temperature (Tm)” is understood to mean a given protein corresponds to the temperature at which 50% of said protein is denatured.

In an embodiment, the polypeptide described herein exhibits a melting temperature (Tm) of from about 45° C. to about 68° C., preferably from about 50° C. to about 65° C., preferably from about 52° C. to about 63° C. In an embodiment, the polypeptide described herein exhibits a melting temperature (Tm) that is lower than the melting temperature (Tm) exhibited by the polypeptide of SEQ ID NO:1. In one embodiment, the polypeptide described herein exhibits a melting temperature (Tm) that is higher than the melting temperature (Tm) exhibited by the polypeptide of SEQ ID NO:1. In an embodiment, the polypeptide described herein exhibits a melting temperature (Tm) of from about 52° C. to about 64° C., preferably from about 55° C. to about 63° C., and more preferably about 63° C.

The present disclosure also extends to polynucleotides comprising a nucleic acid sequence encoding the MHETase polypeptides described herein. This, in an aspect disclosed herein, there is provided a polynucleotide comprising a nucleic acid sequence encoding the polypeptide described herein. In an embodiment, the nucleic acid sequence is selected from the group consisting of SEQ ID NOs:38-72 and 79-84. In another embodiment, the nucleic acid sequence is selected from the group consisting of SEQ ID NOs:79-84. As used herein, the term “nucleic acid”, “nucleic sequence” “polynucleotide”, “oligonucleotide” and “nucleotide sequence” are used interchangeably and refer to a sequence of deoxyribonucleotides and/or ribonucleotides. The nucleic acids can be DNA (cDNA or gDNA), RNA, or a mixture of the two. It can be in single stranded form or in duplex form or a mixture of the two. It can be of recombinant, artificial and/or synthetic origin and it can comprise modified nucleotides, comprising for example a modified bond, a modified purine or pyrimidine base, or a modified sugar. The nucleic acids of the invention can be in isolated or purified form, and made, isolated and/or manipulated by techniques known per se in the art, e.g., cloning and expression of cDNA libraries, amplification, enzymatic synthesis or recombinant technology. The nucleic acids can also be synthesized in vitro by well-known chemical synthesis techniques, as described in, e.g., Belousov (1997) Nucleic Acids Res. 25:3440-3444.

The nucleic acid sequences disclosed herein may suitably be codon optimized. Suitable methods for codon optimization will be familiar to persons skilled in the art, illustrative examples of which are described in the reference manual Sambrook et al. (Sambrook et al., 2001).

The nucleic acid sequences described herein may be suitably deduced from the amino acid sequence of the polypeptides described herein and codon usage may be adapted according to the host cell in which the nucleic acid shall be transcribed.

In some embodiments, the nucleic acid sequences described herein may suitably comprise additional nucleotide sequences, such as regulatory regions, i.e., promoters, enhancers, silencers, terminators, signal peptides and the like that can be used to cause or regulate expression of the polypeptide in a selected host cell or system. Alternatively, or in addition, the nucleic acid sequences described herein may further comprise additional nucleotide sequences encoding fusion proteins, such as maltose binding protein (MBP) or glutathion S transferase (GST) that can be used to favor polypeptide expression and/or solubility.

As noted elsewhere herein, the present disclosure also extends to expression vectors and expression cassettes comprising the nucleic acid sequence described herein, optionally operably linked to one or more control sequences that direct the expression of the nucleic acid sequence in a suitable host cell. Typically, the expression vector or cassette comprises the nucleic acid sequence described herein operably linked to a control sequence such as transcriptional promoter and/or transcription terminator. The control sequence may include a promoter that is recognized by a host cell or an in vitro expression system for expression of the nucleic acid encoding the polypeptide described herein. The promoter will typically comprise a transcriptional control sequence that mediates the expression of the polypeptide. The promoter may be any polynucleotide that shows transcriptional activity in a host cell, including mutant, truncated, and hybrid promoters, and may suitably be obtained from genes encoding extracellular or intracellular polypeptides either homologous or heterologous to the host cell. The control sequence may also be a transcription terminator, which is recognized by a host cell to terminate transcription. The terminator is typically operably linked to the 3′-terminus of the nucleic acid encoding the polypeptide. Any terminator that is functional in the host cell may be used in this context. Typically, the expression vector or cassette comprises the nucleic acid sequence described herein operably linked to a transcriptional promoter and a transcription terminator.

The term “vector” typically refers to a DNA molecule used as a vehicle to transfer recombinant genetic material into a host cell. Suitable vectors include plasmids, bacteriophages, viruses, fosmids, cosmids, and artificial chromosomes. The vector is typically a DNA sequence that comprises an insert (a heterologous nucleic acid sequence, transgene) and a larger sequence that serves as the “backbone” of the vector. The purpose of a vector which transfers genetic information to the host is typically to isolate, multiply, or express the insert in the target cell. Expression vectors (also referred to as expression constructs) are specifically adapted for the expression of the heterologous sequences in the target cell, and generally have a promoter sequence that drives expression of the heterologous sequences encoding a polypeptide.

Generally, the regulatory elements that are used in an expression vector include a transcriptional promoter, a ribosome binding site, a terminator, and optionally present operator. An expression vector may further comprise an origin of replication for autonomous replication in a host cell, a selectable marker, a limited number of useful restriction enzyme sites, and a potential for high copy number. Suitable expression vectors will be familiar to persons skilled in the art, illustrative example of which include cloning vectors, modified cloning vectors, plasmids and viruses. Expression vectors that are capable of providing suitable levels of polypeptide expression in different hosts are also well known in the art. The choice of the vector will typically depend on the compatibility of the vector with the host cell into which the vector is to be introduced. In one embodiment, the vector is the bacterial expression vector pET-28a(+) (SEQ ID NO:85).

The present disclosure also extends to a host cell comprising the nucleic acid sequence described herein. The host cell may be transformed, transfected or transduced in a transient or stable manner. The nucleic acid, expression cassette or vector is introduced into a host cell so that the nucleic acid, cassette or vector is maintained as a chromosomal integrant or as a self-replicating extra-chromosomal vector. The term “host cell” encompasses any progeny of a parent host cell that is not identical to the parent host cell due to mutations that occur during replication. The host cell may be any cell useful in the production of a variant of the present invention, e.g., a prokaryote or a eukaryote. The prokaryotic host cell may be any Gram-positive or Gram-negative bacterium. The host cell may also be a eukaryotic cell, such as a yeast, fungal, mammalian, insect or plant cell. In a particular embodiment, the host cell is selected from the group of Escherichia coli, Pseudomonas, Bacillus, Streptomyces, Trichoderma, Aspergillus, Saccharomyces, Pichia, Thermus or Yarrowia.

The nucleic acid, expression cassette or expression vector according to the invention may be introduced into the host cell by any suitable method known to persons skilled in the art, illustrative examples of which include electroporation, conjugation, transduction, competent cell transformation, protoplast transformation, protoplast fusion, biolistic “gene gun” transformation, PEG-mediated transformation, lipid-assisted transformation or transfection, chemically mediated transfection, lithium acetate-mediated transformation and liposome-mediated transformation.

In an embodiment, the host cell is a genetically modified host cell or microorganism. In this context, a host cell or microorganism may be genetically modified to enhance the expression and/or activity of the polypeptide in which it is expressed. For example, the polypeptide described herein may be used to complement a wild type strain of a fungus or bacterium already known to be capable of MHETase activity, in order to improve and/or increase the MHETase activity of that strain.

The present disclosure also extends to a method of producing a polypeptide having MHETase activity, the method comprising:
a) providing a nucleic acid sequence as described herein;
b) expressing the nucleic acid sequence in a host cell culture, thereby producing the polypeptide; and
c) recovering the polypeptide produced in (b) from the host cell culture.

The present invention disclosure also extends to in vitro methods of producing the polypeptide described herein, the method comprising (a) contacting a nucleic acid, cassette or vector of the invention with an in vitro expression system; and (b) recovering the polypeptide produced. In vitro expression systems are well-known by the person skilled in the art and are commercially available.

Suitable host cells will be familiar to persons skilled in the art, illustrative examples of which include a recombinant Bacillus, recombinant E. coli, recombinant Pseudomonas, recombinant Aspergillus, recombinant Trichoderma, recombinant Streptomyces, recombinant Saccharomyces, recombinant Pichia, recombinant Thermus or recombinant Yarrowia. In an embodiment, the host cell is an E. coli.

The host cells may be cultivated in a nutrient medium suitable for production of polypeptides, using methods that will be known to persons skilled in the art. Suitable examples include cultivating the host cells by shake flask cultivation, or small-scale or large-scale fermentation (including continuous, batch, fed-batch, or solid state fermentations) in laboratory or industrial fermentors performed in a suitable medium and under conditions allowing the enzyme to be expressed and/or isolated. The cultivation will typically take place in a suitable nutrient medium, from commercial suppliers or prepared according to published compositions (e.g., in catalogues of the American Type Culture Collection) or any other culture medium suitable for cell growth. Where the polypeptide is expressed and/or secreted into the nutrient medium, the polypeptide can be used in the form of a cellular/supernatant mixture, or in the form of a crude cell lysate. Alternatively, the polypeptide can be recovered directly from the culture supernatant. Conversely, the polypeptide can be recovered from cell lysates or after permeabilisation of the host cell membrane. The polypeptide may be recovered using any suitable method known to persons skilled in the art, illustrative examples of which include collection, centrifugation, filtration, extraction, spray-drying, evaporation, or precipitation. Optionally, the polypeptide may be partially or totally purified by a variety of procedures known in the art including, but not limited to, thermal chock, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction to obtain substantially pure polypeptides.

The polypeptide may be used, in purified form, either alone or in combination with additional enzymes (e.g., PETases), to catalyze enzymatic reactions involved in the degradation and/or recycling of a polyester containing material, such as plastic products containing polyester. The polypeptides described herein may be in soluble form, or on solid phase. In particular, they may be bound to cell membranes or lipid vesicles, or to synthetic supports such as glass, plastic, polymers, filter, membranes, e.g., in the form of beads, columns, plates and the like.

The present disclosure also extends to compositions comprising the polypeptide, the nucleic acid or the host cell described herein.

The composition may be liquid or dry, for instance in the form of a powder. In some embodiments, the composition is a lyophilisate. For instance, the composition may comprise the polypeptide, nucleic acid and/or host cells and optionally excipients and/or reagents etc. Suitable excipients may include buffers commonly used in biochemistry, agents for adjusting pH, preservatives such as sodium benzoate, sodium sorbate or sodium ascorbate, conservatives, protective or stabilizing agents such as starch, dextrin, arabic gum, salts, sugars e.g., sorbitol, trehalose or lactose, glycerol, polyethyleneglycol, polyethene glycol, polypropylene glycol, propylene glycol, divalent ions such as calcium, sequestering agent such as EDTA, reducing agents (e.g., beta-mercaptoethanol, dithiothreitol, ascorbic acid, tris(2-carboxyethyl)phosphine), amino acids, a carrier such as a solvent or an aqueous solution, and the like.

In an embodiment, the composition comprises the polypeptide described herein (the polypeptide may be present in the composition in an isolated or at least partially purified form). In an embodiment, the composition comprises the polypeptide described herein in an amount of from about 0.1% to about 99.9%, preferably from about 0.1% to about 50%, preferably from about 0.1% to about 30%, preferably from about 0.1% to about 5% by weight of the total weight of the composition. In a preferred embodiment, the composition comprises the polypeptide described herein in an amount of from about 0.1 to about 5% by weight of the total weight of the composition. In another embodiment, the composition comprises the polypeptide described herein in an amount of from about 0.1 to about 0.2% by weight of the total weight of the composition. The amount of polypeptide in the composition may suitably adapted by persons skilled in the art, depending e.g., on the nature and/or amount of the polyester containing material to be degraded (hydrolysed) and/or the presence or absence of any additional enzymes/polypeptides in the composition.

The compositions described herein may further comprise additional polypeptide(s) exhibiting enzymatic activity, not limited to MHETases.

In an embodiment, the polypeptide described herein is solubilized in an aqueous medium together with one or more excipients, such as excipients that may suitable stabilize or protect the polypeptide from degradation. For example, the polypeptides described herein may be solubilized in water and then admixed with excipients such as glycerol, sorbitol, dextrin, starch, glycol such as propanediol, salt, etc. The resulting admixture may then be dried so as to obtain a powder. Methods for drying such mixture are well known to the one skilled in the art and include, without limitation, lyophilisation, freeze-drying, spray-drying, supercritical drying, down-draught evaporation, thin-layer evaporation, centrifugal evaporation, conveyer drying, fluidized bed drying, drum drying or any combination thereof.

In an embodiment, the composition comprises at least one host cell expressing the polypeptide described herein, or an extract thereof. By “extract of a cell” is meant any fraction obtained from a cell, such as cell supernatant, cell debris, cell walls, DNA extract, enzymes or enzyme preparation or any preparation derived from cells by chemical, physical and/or enzymatic treatment, which is essentially free of living cells. Preferred extracts are enzymatically-active extracts. The composition may comprise one or several host cells or extract thereof containing the polypeptide described herein, and optionally one or several additional cells.

As noted elsewhere herein, the present inventors have surprisingly found that the polypeptides described herein (the MHETase variants) have greater MHETase activity when compared to the wild-type MHETase of SEQ ID NO:1. Thus, disclosed herein is a method of hydrolysing a mono-(2-hydroxyethyl) terephthalate, the method comprising exposing the mono-(2-hydroxyethyl) terephthalate to the polypeptide, the composition or the host cell described herein, under conditions sufficient to convert the mono-(2-hydroxyethyl) terephthalate to terephthalate and ethylene glycol. The present disclosure also extends to a method of degrading a plastic product comprising a polyester, the method comprising exposing the plastic product to the polypeptide, the composition or the host cell described herein.

The present disclosure extends to the use the polypeptide, the composition or the host cell described herein for degrading a polyester in aerobic or anaerobic conditions and/or recycling polyester containing material, as plastic products made of or containing polyesters and/or producing biodegradable plastic products containing polyester. Such methods and used are particularly useful for degrading a plastic product comprising PET.

Advantageously, the polyester(s) of the polyester containing material is (are) depolymerized up to monomers and/or oligomers. In an embodiment, at least one polyester is degraded to yield re-polymerizable monomers and/or oligomers, which are advantageously retrieved or recovered for further use.

In an embodiment, polyester(s) of the polyester containing material is (are) fully degraded.

As noted elsewhere herein, the plastic product may comprise at least one polyester selected from the group consisting of polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA) and combinations of any of the foregoing.

The time required for degrading a polyester containing material may vary depending on the polyester containing material itself (i.e., nature and origin of the plastic product, its composition, shape etc.), the type and amount of polypeptide used, as well as various process parameters (i.e., temperature, pH, additional agents, etc.). One skilled in the art may easily adapt the process parameters to the polyester containing material.

Advantageously, the degrading process is implemented at a temperature from about 10° C. to about 60° C., preferably from about 20° C. to about 60° C., preferably from about 30° C. to about 60° C., more preferably from about 40° C. to about 60° C., even more preferably from about 40° C. to about 50° C., or even more preferably at about 45° C. The temperature is typically be maintained below an inactivating temperature, which corresponds to the temperature at which the polypeptide is inactivated and/or the recombinant microorganism does not synthesize, produce or release the polypeptide described herein. In an embodiment, the temperature is maintained below the glass transition temperature (Tg) of the polyester in the polyester containing material. In an embodiment, the degrading process or method is implemented at a temperature from about 10° C. to about 60° C., preferably from about 20° C. to about 60° C., preferably from about 30° C. to about 60° C., more preferably from about 40° C. to about 60° C., even more preferably from about 40° C. to about 50° C., or even more preferably at about 45° C. The process or method may suitably be implemented in a continuous way, at a temperature at which the polypeptide can be used several times and/or recycled.

Advantageously, the degrading process or method is implemented at a pH comprised between 5 and 11, preferably at a pH between 6 and 10, more preferably at a pH between 6.5 and 9, even more preferably at a pH between 7 and 8.

In an embodiment, the polyester containing material may be pretreated prior to be contacted with the polypeptide in order to physically change its structure, so as to increase the surface of contact between the polyester and the enzyme.

Monomers resulting from the depolymerization or degradation process or method may be suitably recovered, sequentially or continuously. A single type of monomers or several different types of monomers may be recovered, depending on the starting polyester containing material.

The recovered monomers may be further purified, using any suitable purifying method and conditioned in a repolymerizable form. Illustrative examples of suitable purifying methods include stripping process, separation by aqueous solution, steam selective condensation, filtration and concentration of the medium after the bioprocess, separation, distillation, vacuum evaporation, extraction, electrodialysis, adsorption, ion exchange, precipitation, crystallization, concentration and acid addition dehydration and precipitation, nanofiltration, acid catalyst treatment, semi continuous mode distillation or continuous mode distillation, solvent extraction, evaporative concentration, evaporative crystallization, liquid/liquid extraction, hydrogenation, azeotropic distillation process, adsorption, column chromatography, simple vacuum distillation and microfiltration, combined or not.

The repolymerizable monomers may be used to synthesize new polyesters. Advantageously, polyesters of same nature are repolymerized. However, it is possible to mix the recovered monomers with other monomers, for example, in order to synthesize new copolymers. Alternatively, the recovered monomers may be used as chemical intermediates in order to produce new chemical compounds of interest.

The present disclosure also extends to a plastic compound comprising the polypeptide, composition and/or host cell expressing said polypeptide or an extract thereof containing said polypeptide.

The present disclosure also extends to a masterbatch composition comprising the polypeptide, composition and/or host cell expressing said polypeptide or an extract thereof containing said polypeptide.

Advantageously, such plastic compound or masterbatch composition described herein can be used for the production of a polyester containing material and/or plastic article that will include the polypeptide described herein.

In an embodiment, the resulting plastic compound, masterbatch composition or plastic article is a biodegradable plastic compound, masterbatch composition or plastic article complying with at least one of the relevant standards and/or labels known by the person skilled in the art, such as standard EN 13432, standard ASTM D6400, OK Biodegradation Soil (Label Vincotte), OK Biodegradation Water (Label Vincotte), OK Compost (Label Vincotte), OK Home Compost (Label Vincotte).

Advantageously, the degrading process of the polyester containing material (i.e., plastic compound, masterbatch composition or plastic product) is implemented at a temperature comprised between 10° C. and 50° C., preferably between 15° C. and 40° C., more preferably between 20° C. and 30° C., more preferably at 28° C., +/−2° C.

Alternatively, the degrading process of the polyester containing material (i.e., plastic compound, masterbatch composition or plastic product) is implemented at a temperature comprised between 50° C. and 60° C., more preferably at 55° C., +/−2° C.

The MHETase variants disclosed herein are suitable for a range of application, including industrial applications, illustrative examples of which include as additives in detergents, feed compositions (including for animal feed), textiles production, electronics and biomedical applications. For example, the MHETase variants disclosed herein can be employed in textile production, where it can be used as an exonuclease to suitably modify the properties of textile fibres.

The invention will now be described with reference to the following Examples which illustrate some preferred aspects of the present invention. However, it is to be understood that the particularity of the following description of the invention is not to supersede the generality of the preceding description of the invention

### EXAMPLES

### Materials and Methods

### Construction, Expression and Purification of Recombinant Mhetases

### A. Consensus Design Construction

5,000 sequences were collected by BLAST+ using wild-type I. sakasiensis MHETase (SEQ ID NO:1) as a seed sequence, an E-value threshold of 10-5 was used.

(UniProt Accession No. A0A0K8P8E7)
SEQ ID NO: 1
GGGSTPLPLPQQQPPQQEPPPPPVPLASRAACEALKDGNGDMVWPNAATV
VEVAAWRDAAPATASAAALPEHCEVSGAIAKRTGIDGYPYEIKFRLRMPA
EWNGRFFMEGGSGTNGSLSAATGSIGGGQIASALSRNFATIATDGGHDNA
VNDNPDALGTVAFGLDPQARLDMGYNSYDQVTQAGKAAVARFYGRAADKS
YFIGCSEGGREGMMLSQRFPSHYDGIVAGAPGYQLPKAGISGAWTTQSLA
PAAVGLDAQGVPLINKSFSDADLHLLSQAILGTCDALDGLADGIVDNYRA
CQAAFDPATAANPANGQALQCVGAKTADCLSPVQVTAIKRAMAGPVNSAG
TPLYNRWAWDAGMSGLSGTTYNQGWRSWWLGSFNSSANNAQRVSGFSARS
WLVDFATPPEPMPMTQVAARMMKFDFDIDPLKIWATSGQFTQSSMDWHGA
TSTDLAAFRDRGGKMILYHGMSDAAFSALDTADYYERLGAAMPGAAGFAR
LFLVPGMNHCSGGPGTDRFDMLTPLVAWVERGEAPDQISAWSGTPGYFGV
AARTRPLCPYPQIARYKGSGDINTEANFACAAPP

315 non-redundant sequences showed high similarity to MHETase and were retrieved from the UniProt database. Peptide transport signals were identified using SignalP4.0 and deleted. All the sequences were aligned using the PROMALS3D library-based sequence alignment algorithm and the available MHETase structure (6QGB), before the final curated alignment was manually refined. A consensus of the alignment at each amino acid position was constructed using a number of different thresholds (95%, 90%, 85%, 80%, 75%, 70%, 65%, 60%, 55%, and 50%). A truncated codon-optimised version of MHETase gene (GenBank Accession No. A0A0K8P8E7), lacking the first 19 amino acids at the N′ terminal as well as the 10 consensus designs were commercially synthesized and cloned into pET-28a (+) (Twist Bioscience).

### B. Protein Expression and Purification

Plasmids were transformed by electroporation into electrocompetent E. coli SHuffle T7 Express cells (New England Biolab) and plated onto Lysogeny broth (LB) agar supplemented with 100 μg/mL ampicillin and incubated at 37° C. overnight. A single colony was used to inoculate 10 ml of LB supplemented with 100 μg/mL ampicillin (LBA) and incubated at 30° C. overnight. This culture was added to 1 L of LBA and incubated at 30° C. until the OD600 reached 1.0. A final concentration of 1 mM Isopropyl β-D-1-thiogalactopyranoside was added and the cultures were transferred to 18° C. incubation for 16 hours.

Cells were harvested by centrifugation at 5000×g for 15 minutes at 4° C. and resuspending in Lysis Buffer (500 mM NaCl, 30 mM Imidazole, 0.5 mg/mL Lysozyme, 1% (v/v) TritonX-100, 1 U/ml Turbonuclease (Sigma), 0.5 mM Dithiothreitol (DTT) and 25 mM HEPES pH 7.5). The cell suspension was lysed by two rounds of sonication at 50% power and pulse time for 5 mins and soluble cell lysate was separated from the insoluble cell debris by centrifugation at 32,000×g for 45 minutes at 4° C. The lysate was passed through a 0.45 μm pore size filter and then purified by nickel-charged IMAC using a 5 mL HisTrap HP (GE Healthcare Life Sciences) equilibrated in Lysis Buffer and eluted off with Elution Buffer (500 mM NaCl, 500 mM Imidazole, 0.5 mM Dithiothreitol (DTT) and 25 mM HEPES pH 7.5). The elution associated with MHETase was collected, concentrated and filtered through a 0.2 μm filter. The filtered product was further purified using a HiLoad 26/600 Superdex 200 (GE Healthcare Life Sciences) equilibrated in SEC Buffer (150 mM NaCl, 25 mM HEPES pH 7.5).

### C. Chromogenic Assay

50 μL of cell suspension of expressed protein was diluted in 150 μL of in Reaction Buffer (90 mM NaCl, 45 mM Sodium Phosphate pH 7.5). The reaction was initiated by the addition of 50 μL of 10 mM 1NT (1-Naphthyl Terephthalate) and 5 mM Fast Blue B Salt Dye in 100% (v/v) DMSO. The absorbance at 465 nm was monitored for 30 minutes using the Epoch Microplate Spectrophotometer (BioTek).

For kinetics determination, a final concentration of 7.5 nM homogeneous MHETase was used instead of soluble cell lysate along varying concentrations of 1NT from 1 mM to 7.8 μM. The concentration of Fast Blue B salt remained constant. The absorbance was converted to product concentration by using a calibration curve of 1 mM to 7.8 μM of 1-Naphthol.

### D. SDS PAGE Atto550

Methods were adapted from Raducanu et al. (2020, Journal of Biological Chemistry 295(34):12214-12223). 1 mL of cell pellet was resuspended in 1× BugBuster (Merck-Millipore) diluted in SEC Buffer and incubated at room temperature for 10 mins. The mixture was centrifuged at 15,000×g for 10 mins and 5 μL of soluble cell lysate was run on an SDS-PAGE gel for 60 mins at 140 V. The gel was microwaved for 30 sec twice in Milli-Q water (MQ) then microwaves in Fixing Solution (40% (v/v) methanol, 10% (v/v) acetic acid in MQ) for 2 mins. The now fixed protein gel was microwaved again for 10 mins in MQ and the incubated for 1 hour on a shaker in a dark environment in a 1:3000 dilution of NTA-Atto550 dye in PBS buffer. The gel was then transferred to a container of warm MQ for an additional 30 mins of shaking. The gel was then imaged using the ChemiDoc MP Imaging System (BIO-RAD) using the DyLight 550 fluorophore option.

### E. Circular Dichroism and Thermal Stability

Measurement of the circular dichroism spectra for MHETase was performed in a 1 mm quartz cuvette on the Applied Photophysics Chirascan Spectrometer. The homogeneous enzyme was diluted to 0.2 mg/mL in 25 mM Sodium Acetate pH 7.5. The CD spectra was measured at 20° C. between 200 nm to 260 nm, a bandwidth of 1 nm and a scan rate of 0.5 seconds was used with adaptive sampling enabled. The spectra were measured in triplicate and a buffer blank was subtracted from the results. For the assessment of thermal melt, the CD at 222 nm was recorded as the temperature of the solution was increased from 20° C. to 90° C., 1° C./min. A standard sigmoidal curved was fitted to the thermal melt data to determine the Tm.

### F. High-Performance Liquid Chromatography (HPLC) Activity Assessment for MHETase

HPLC assay was adapted from Palm et al. (2019). Homogeneous MHETase was diluted in reaction buffer to a final concentration of 7.5 nM (80 μL). The reaction was initiated by adding 20 μL of 1 mM MHET dissolved in 100% DMSO. The reaction was quenched after set time points (0, 10, 30 and 60 mins) by adding 100 μL Quenching Buffer (160 mM Sodium Phosphate pH 2) and heated to 80° C. for 10 mins. A volume of 10 μL of the reaction mix was loaded onto an Agilent ZORBAX SB-C18, 3.5 um, 4.6×150 mm column. TPA and MHET were separated using a flow rate of 1 mL/min at 30° C. equilibrated in 50% Phosphate Buffer (20 mM Sodium Phosphate pH 2.0) and 50% Acetonitrile over a 7-minute run time. The TPA and MHET were detected at 240 nm and quantified against a calibration curve.

### Example 1: Variant MHETases

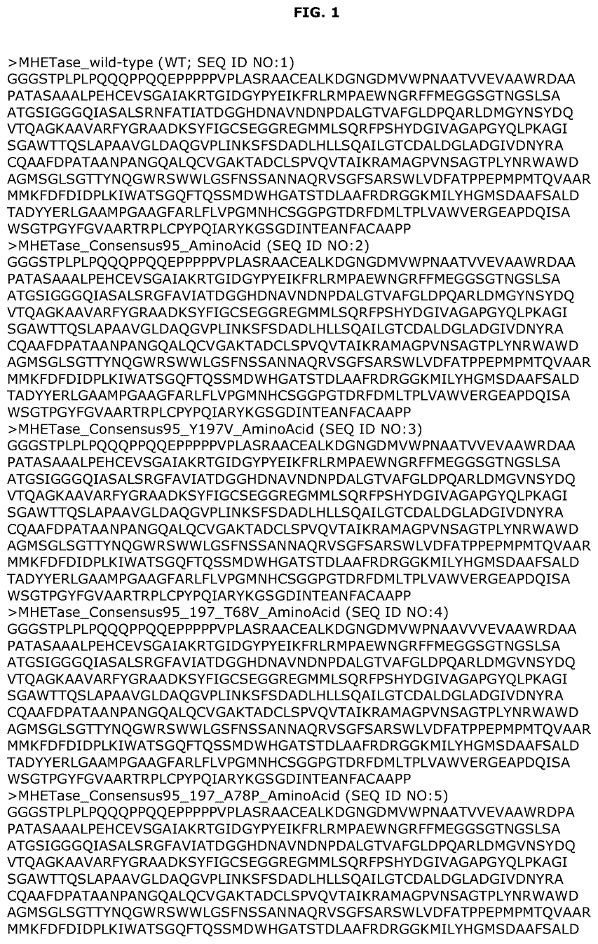

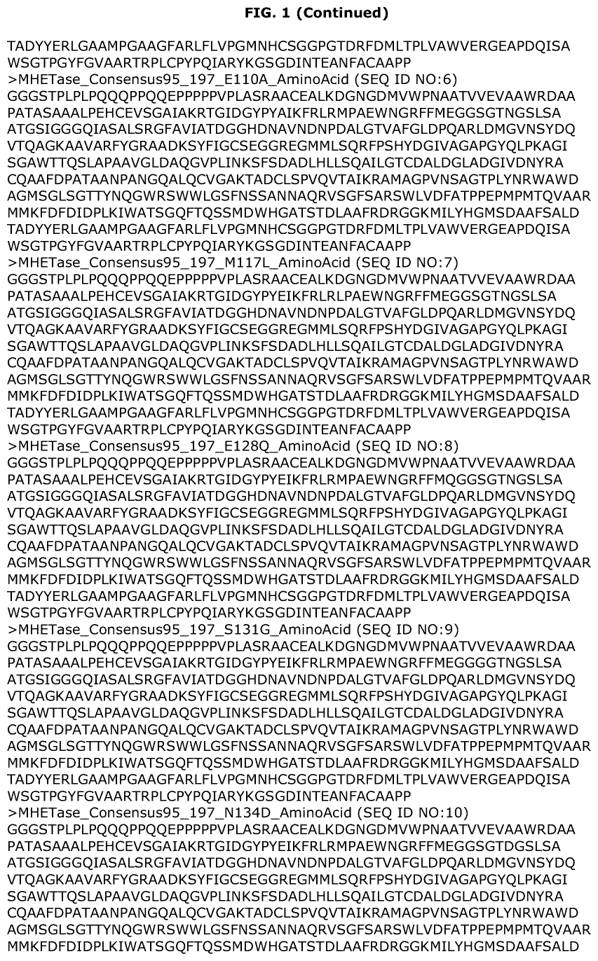

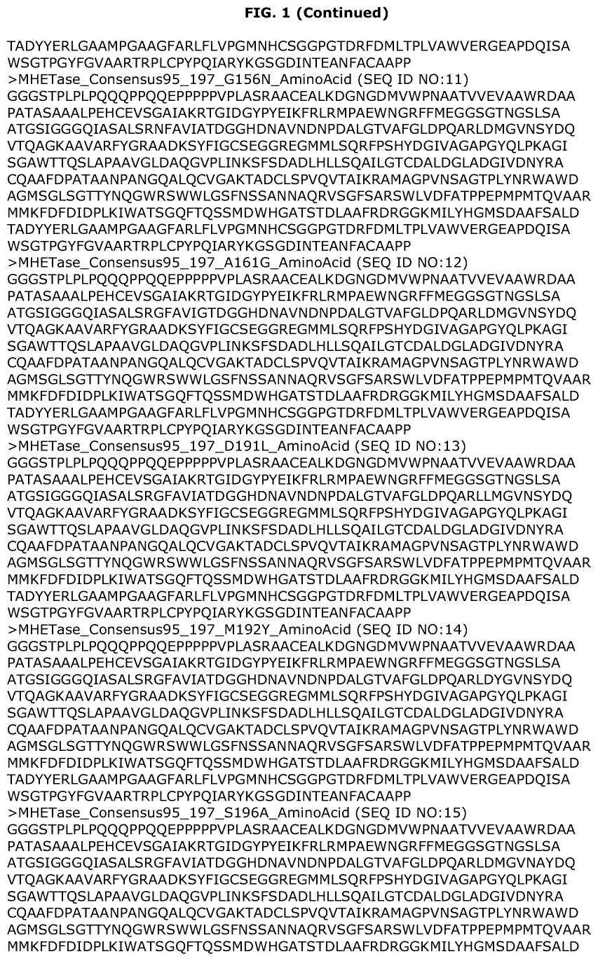

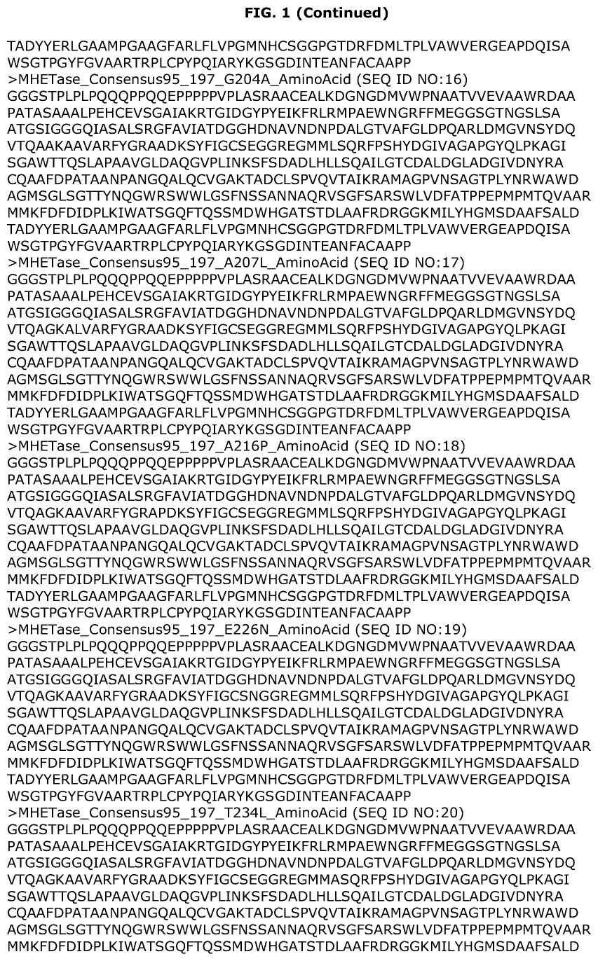

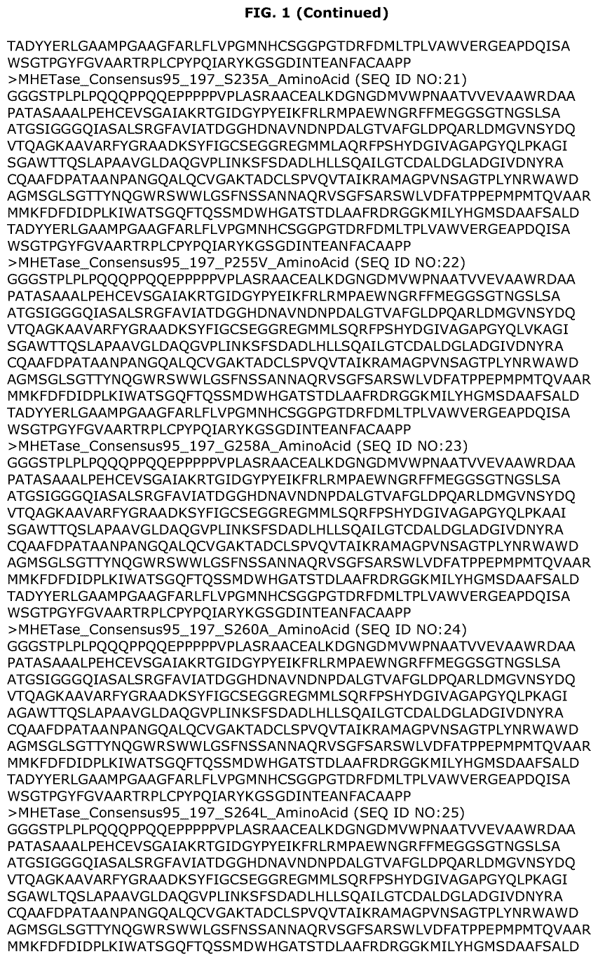

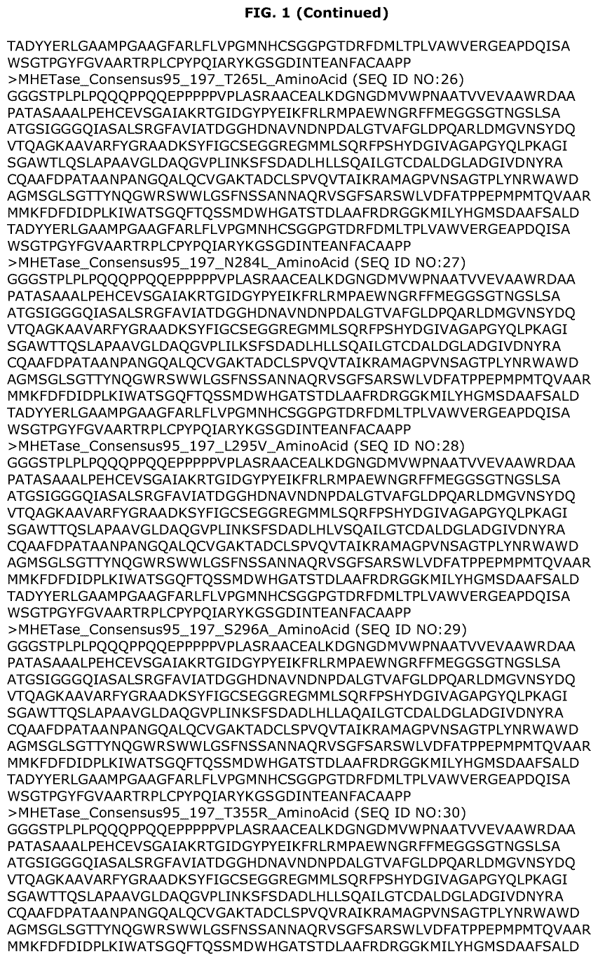

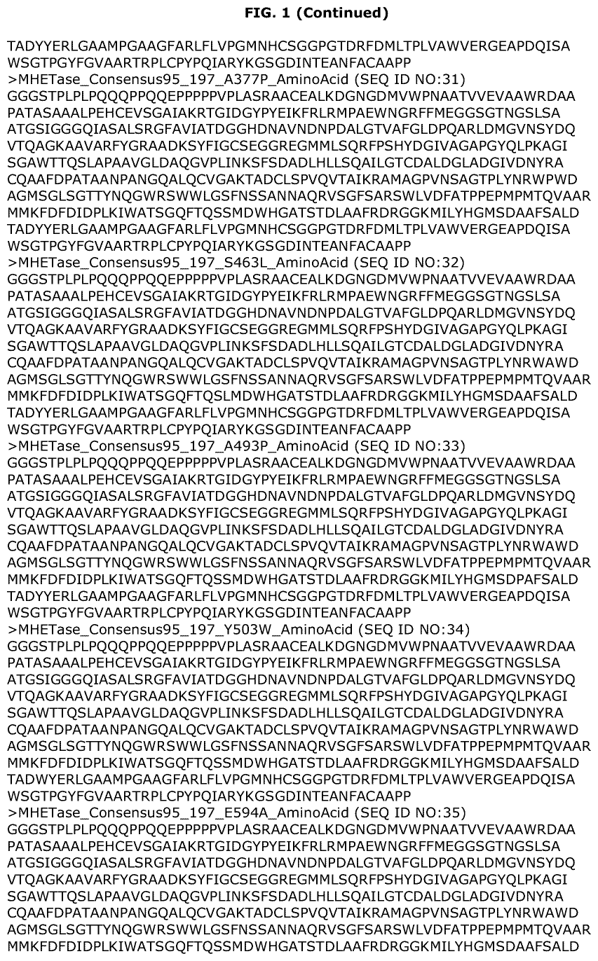

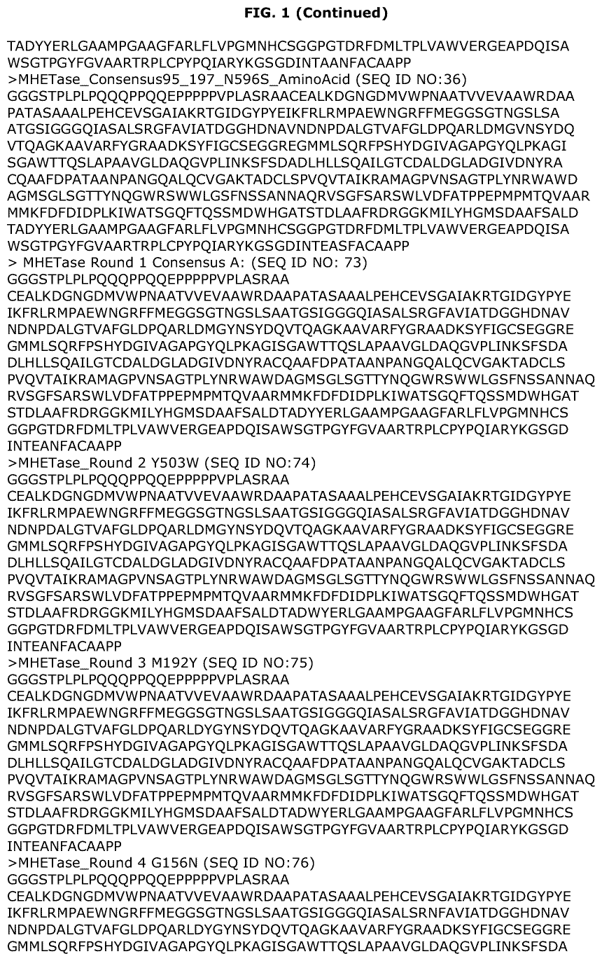

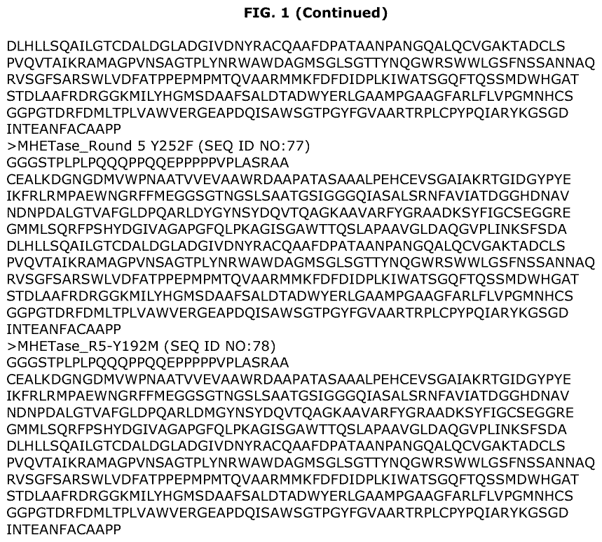

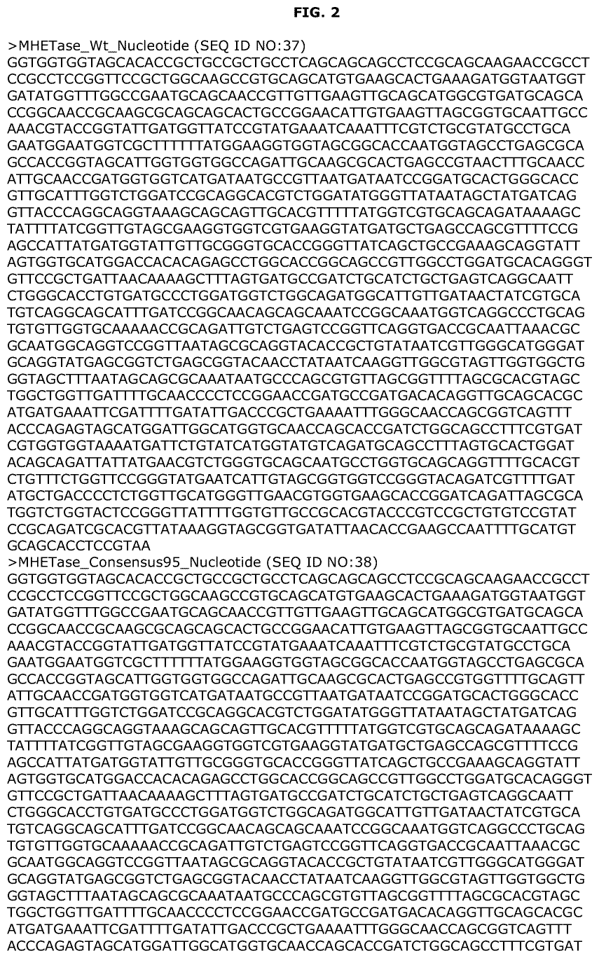

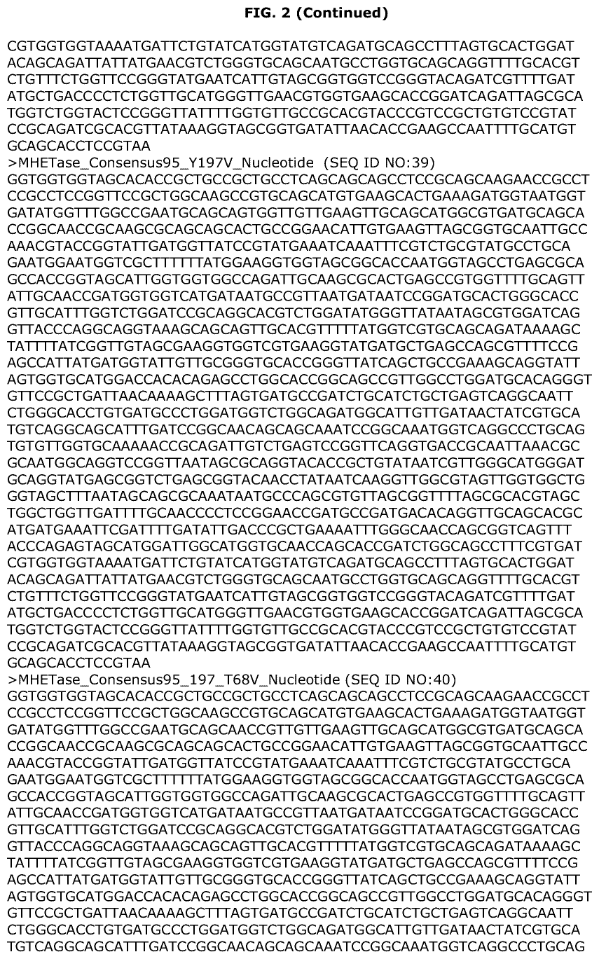

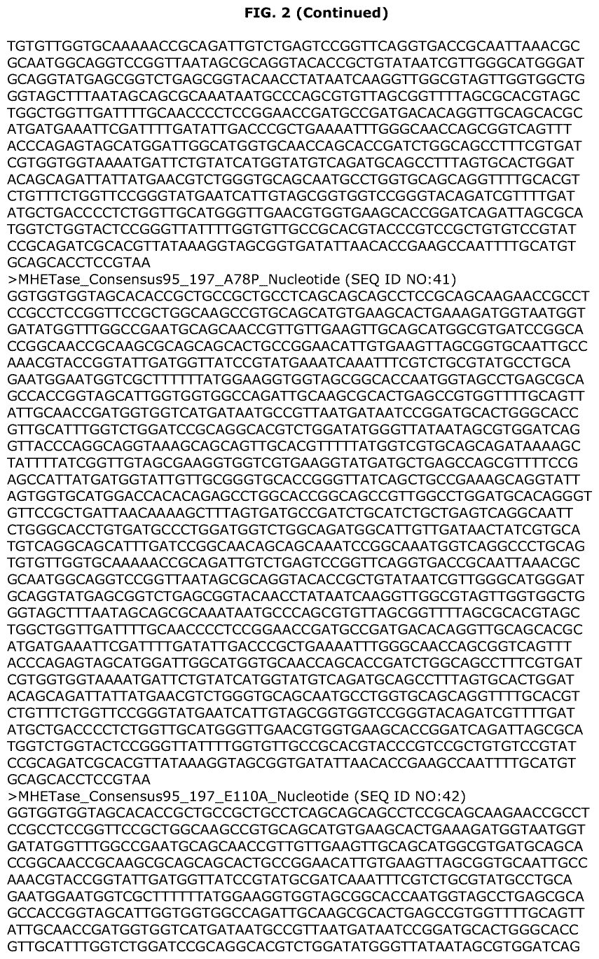

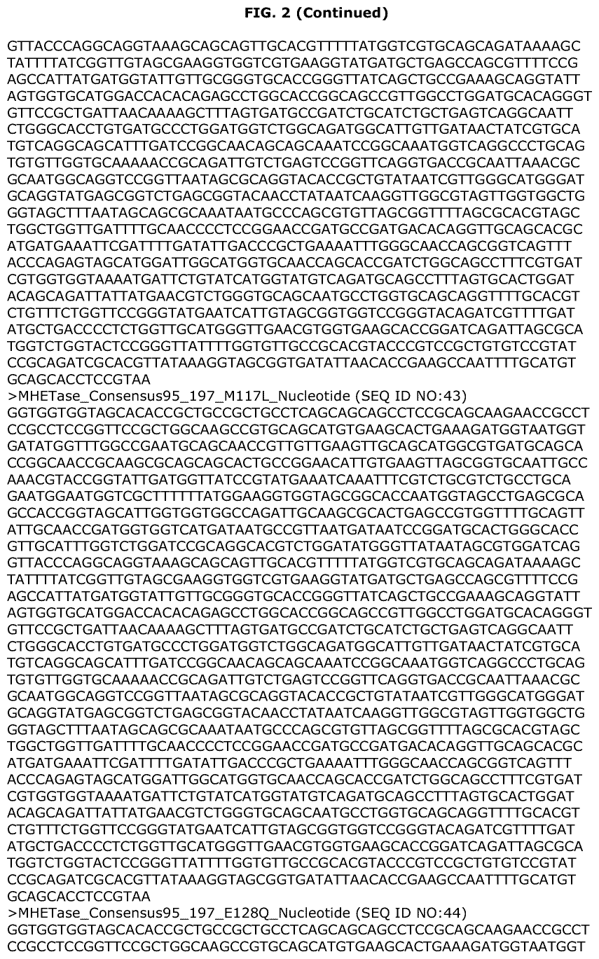

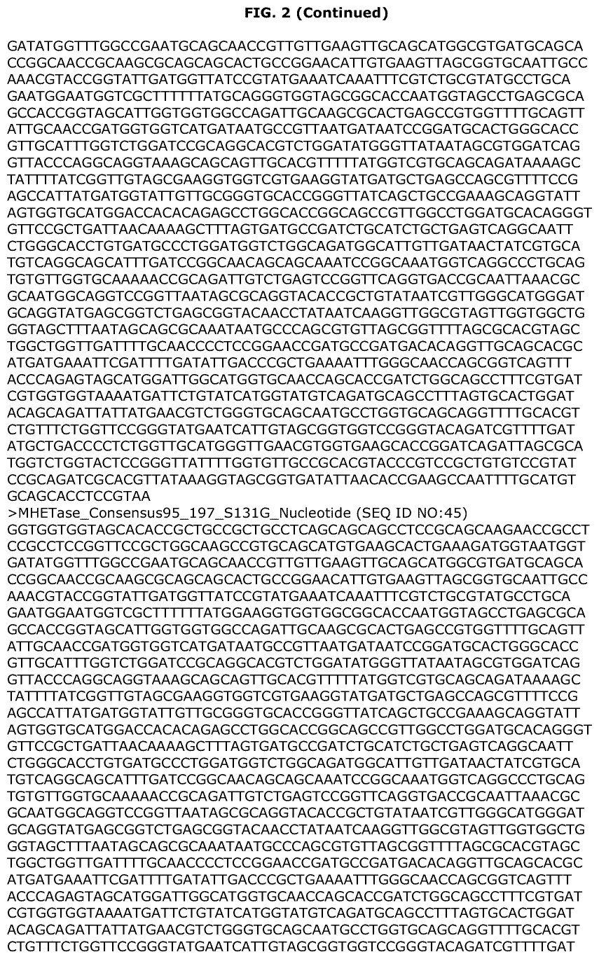

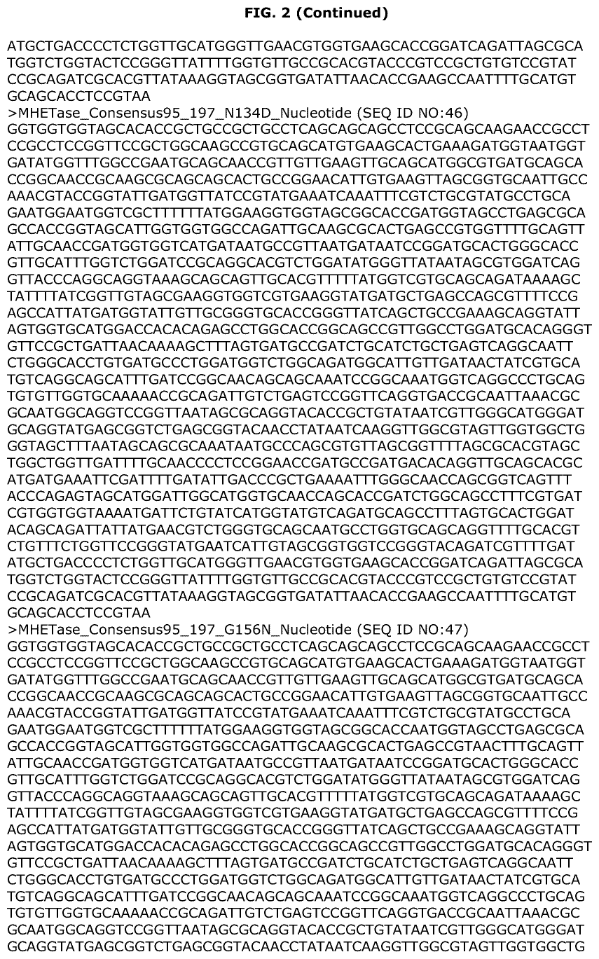

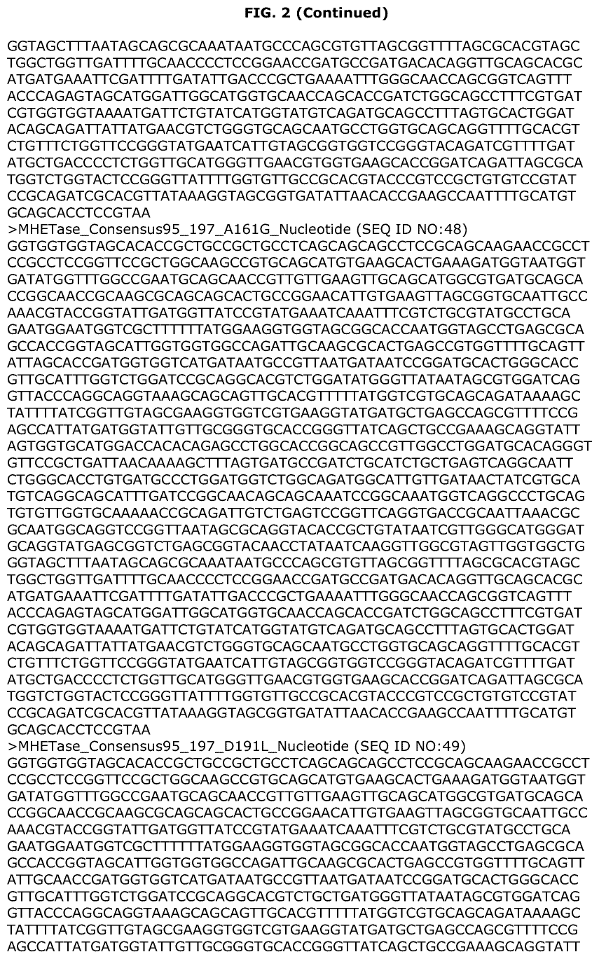

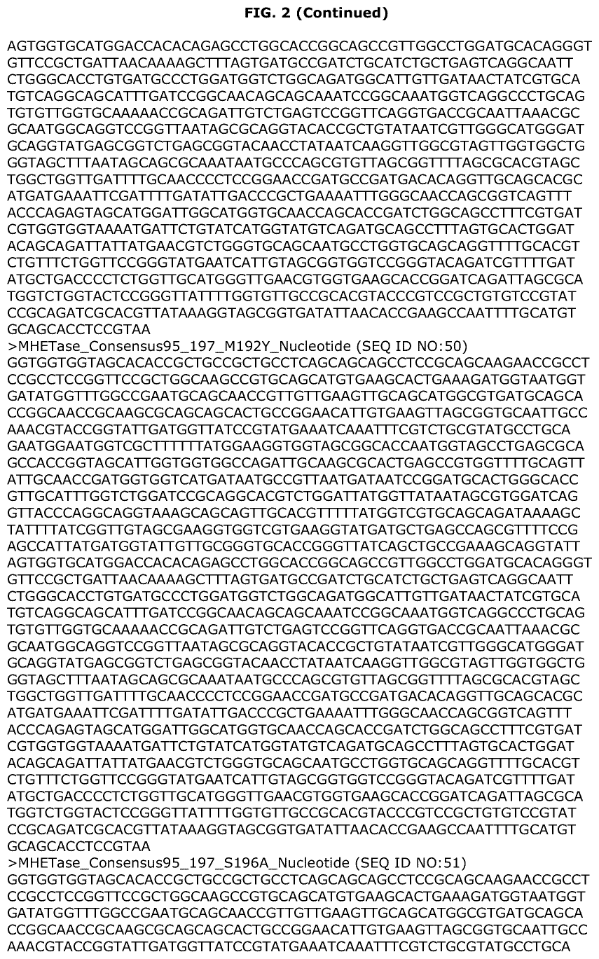

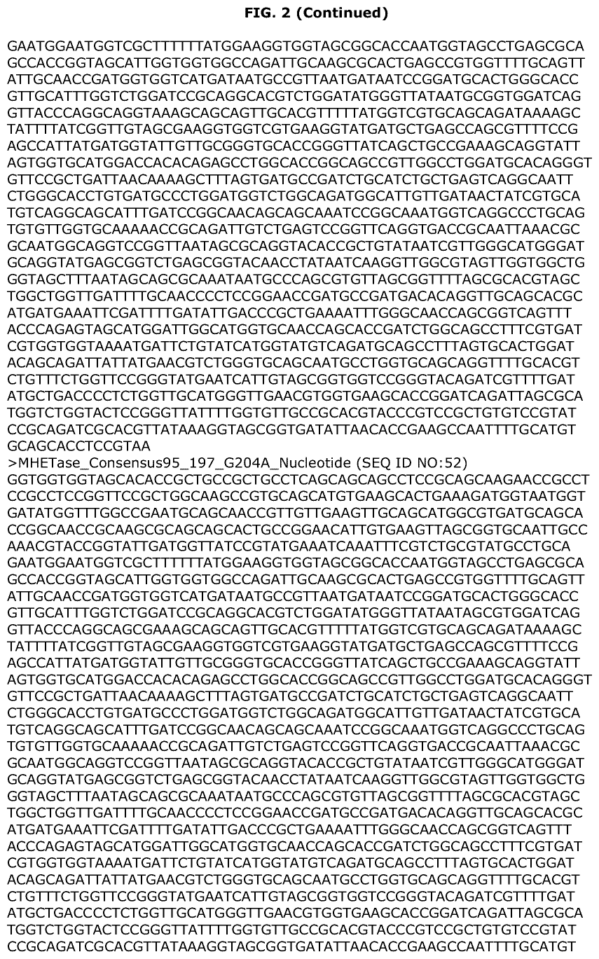

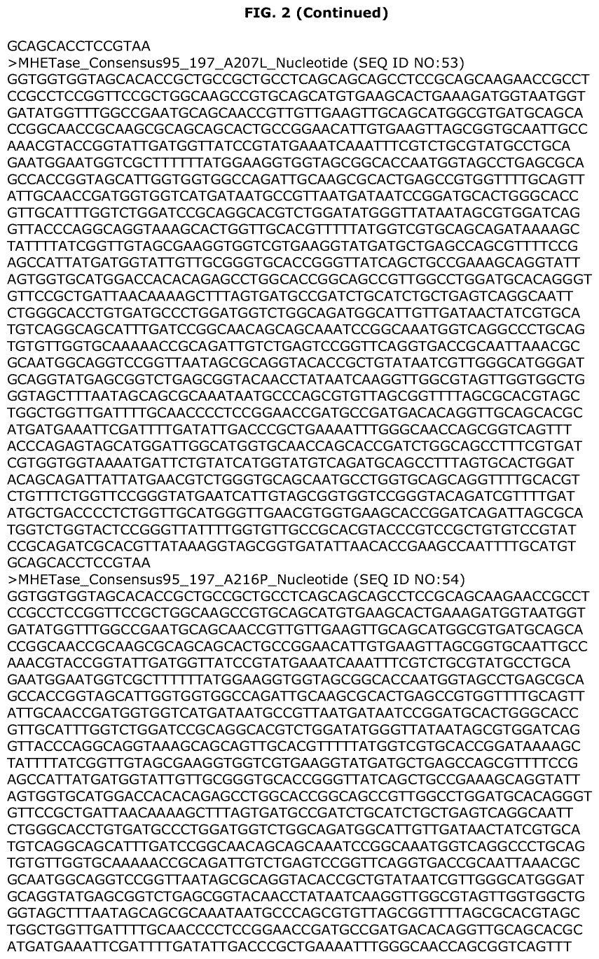

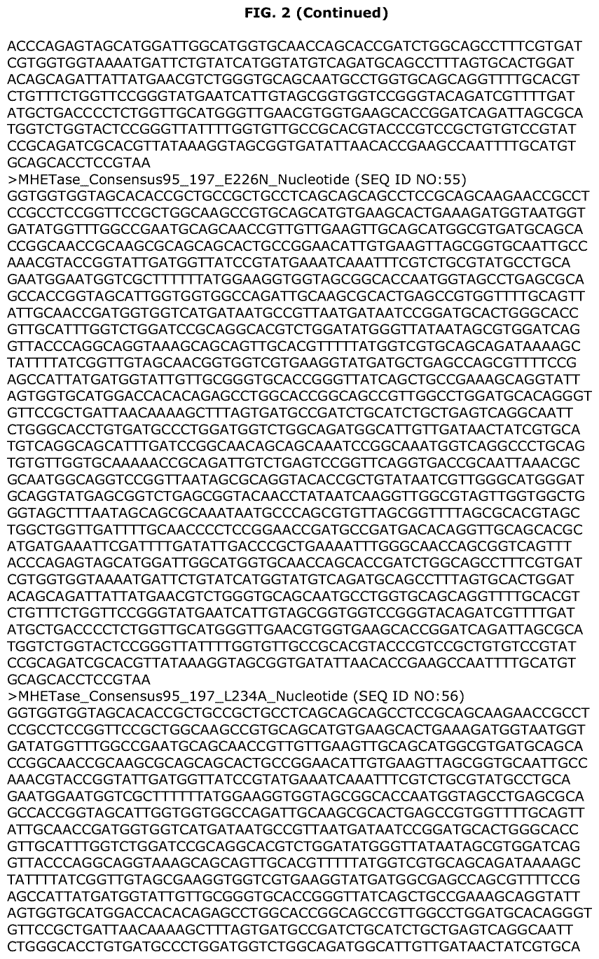

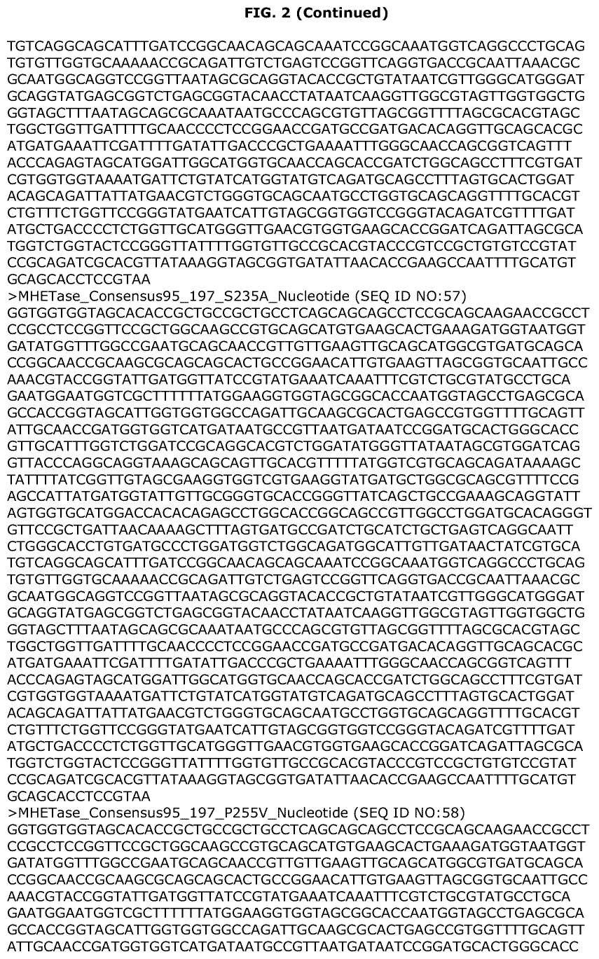

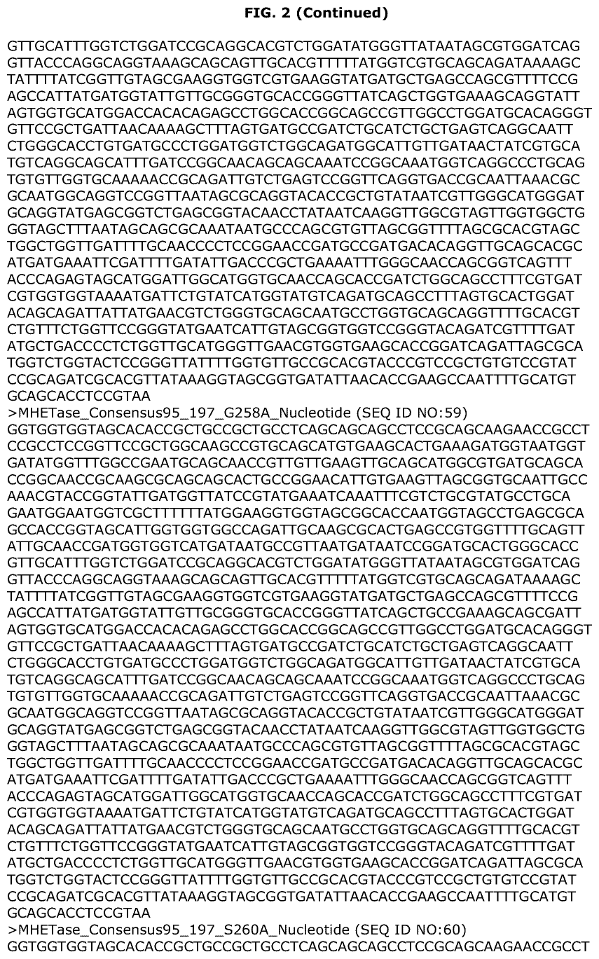

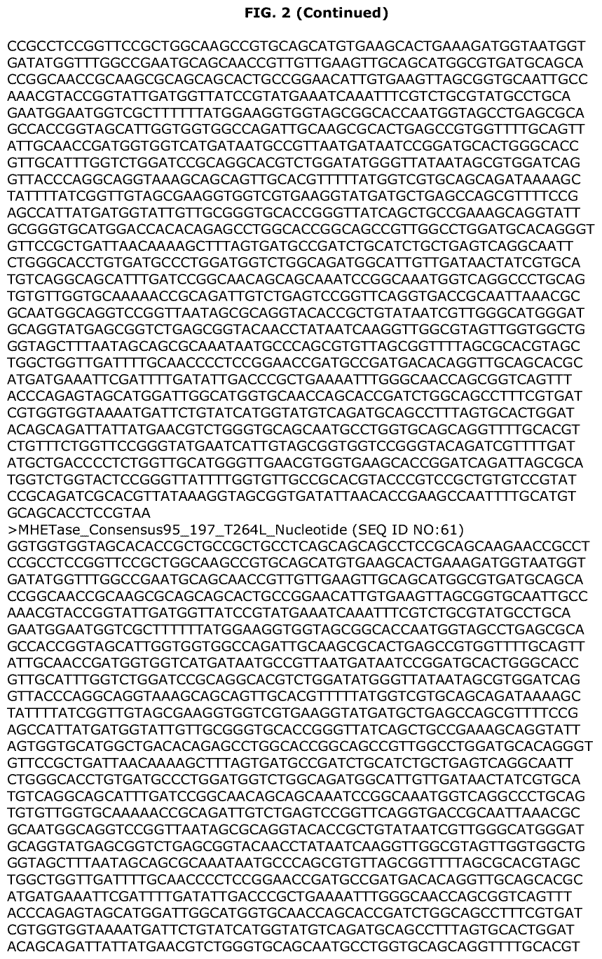

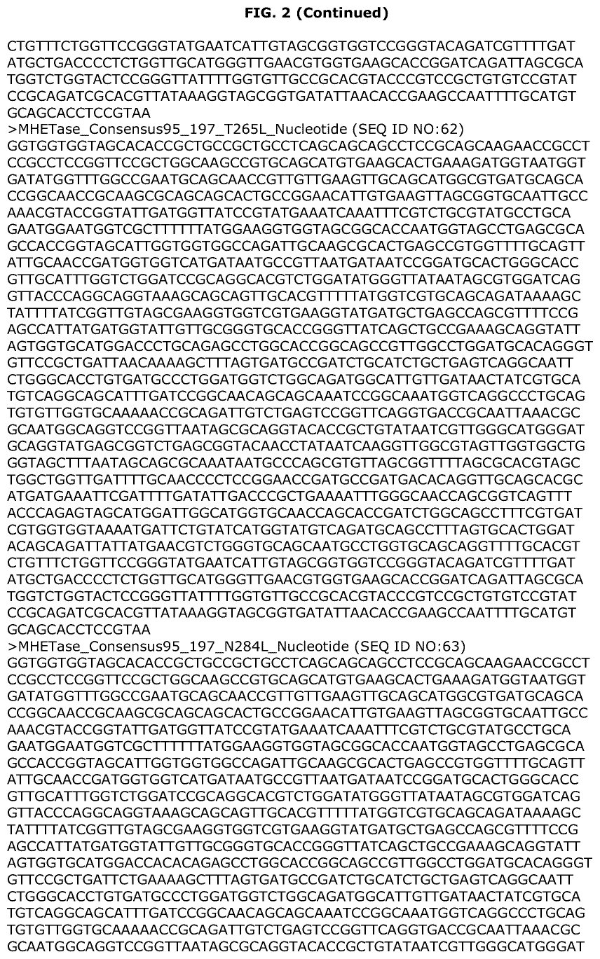

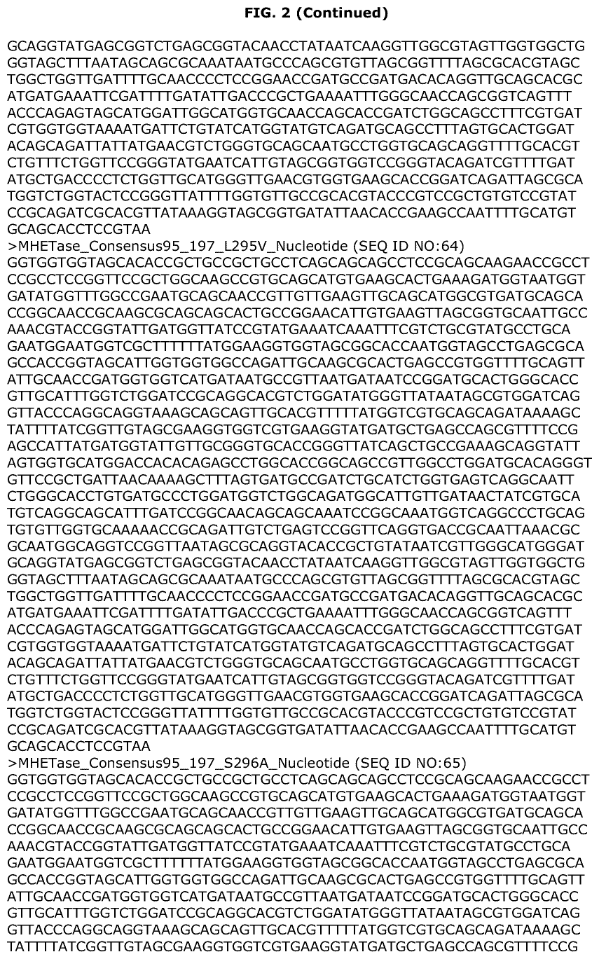

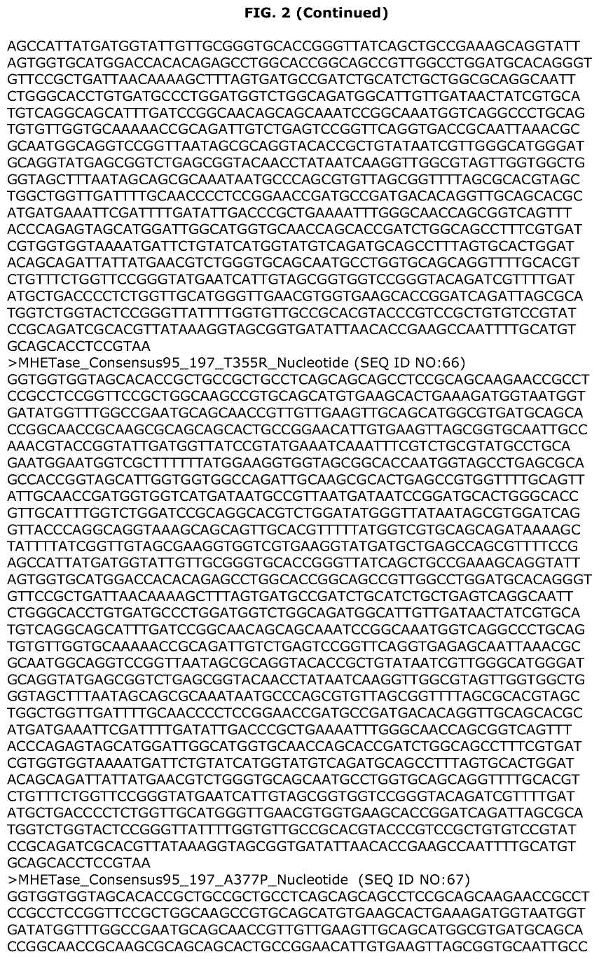

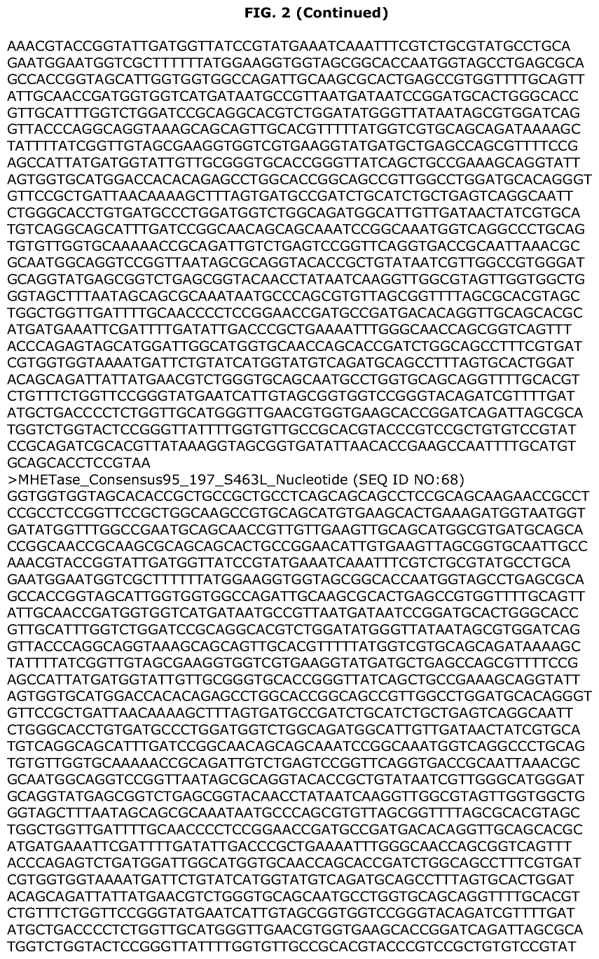

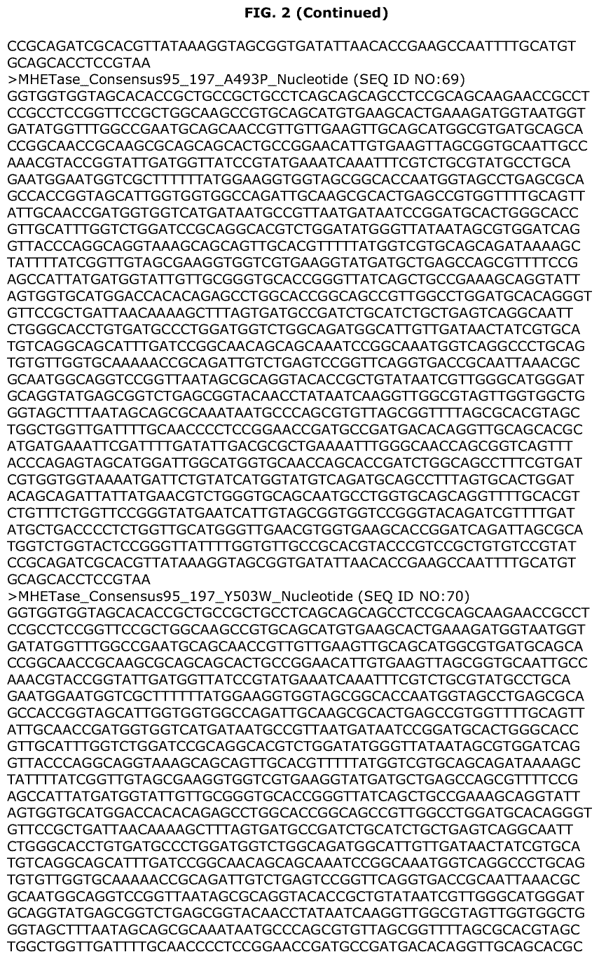

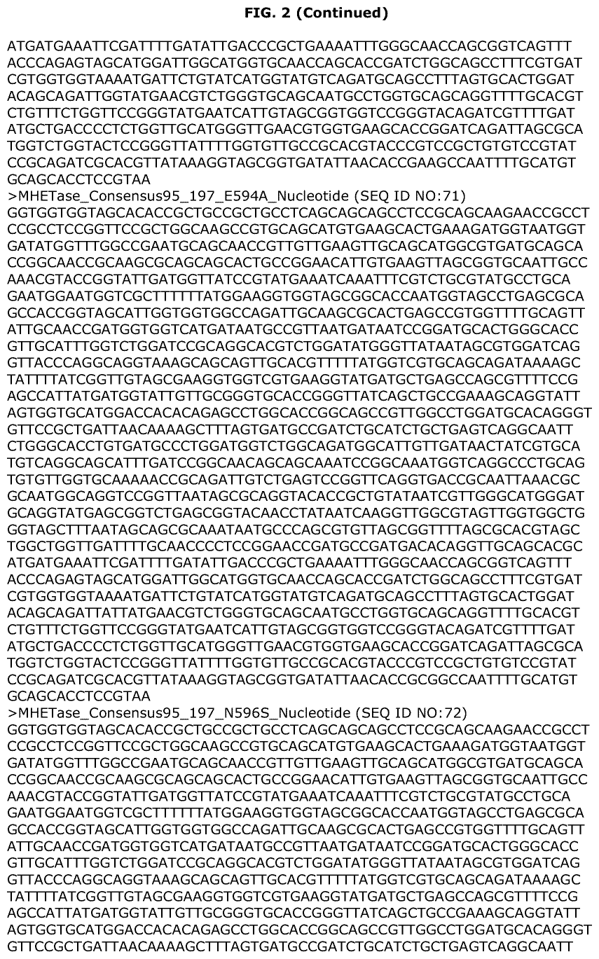

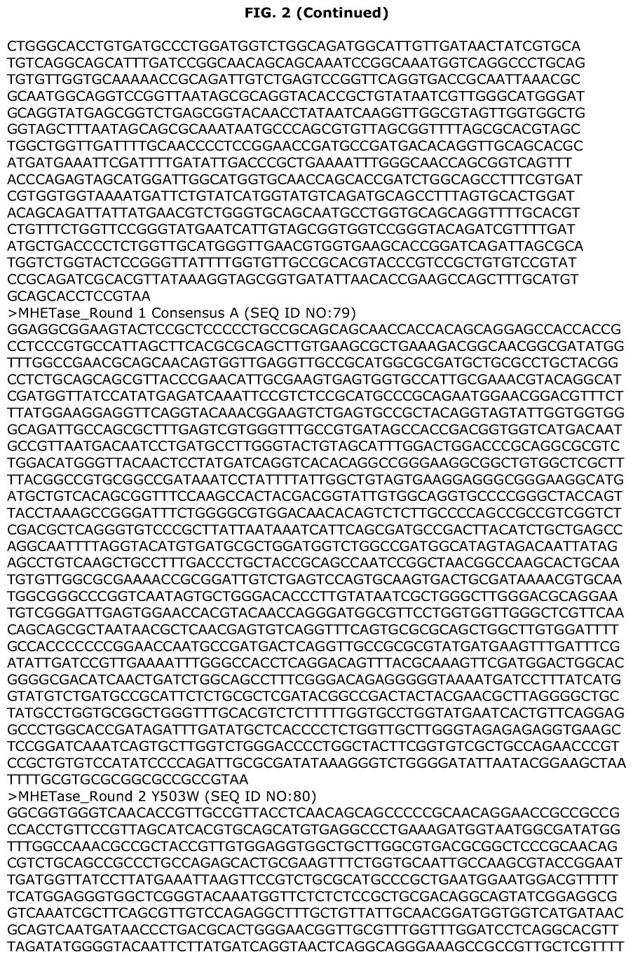

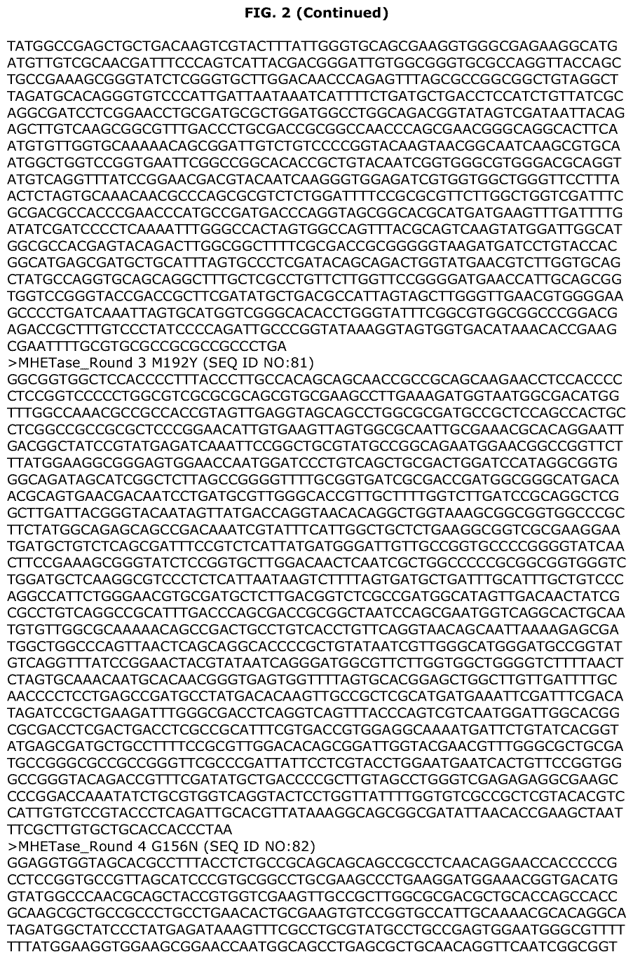

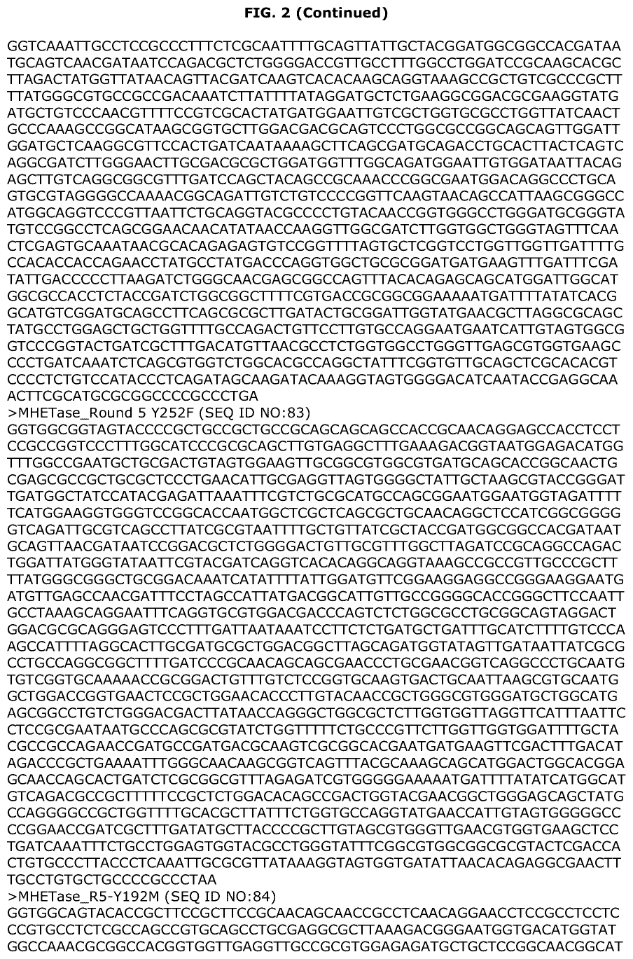

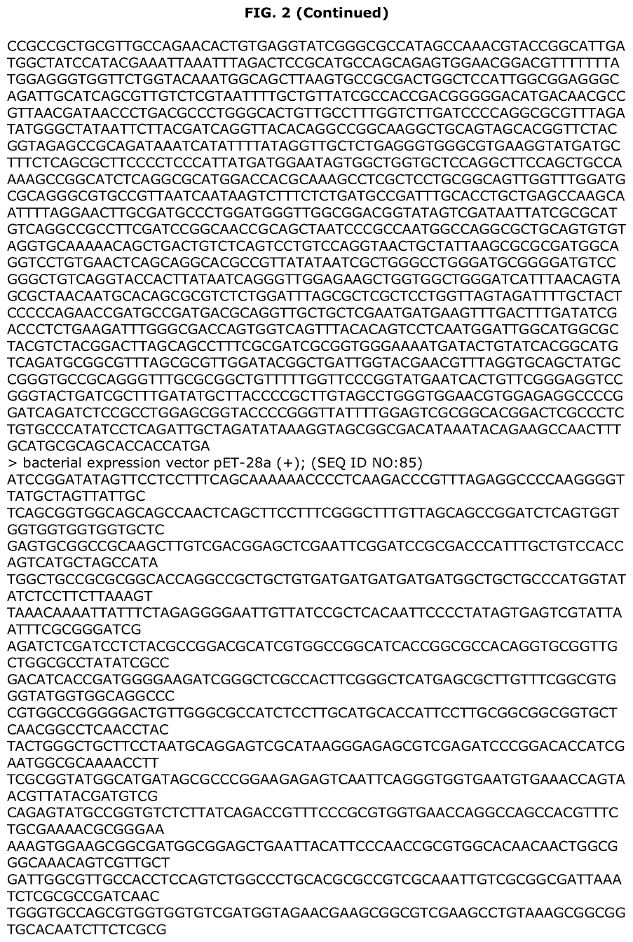

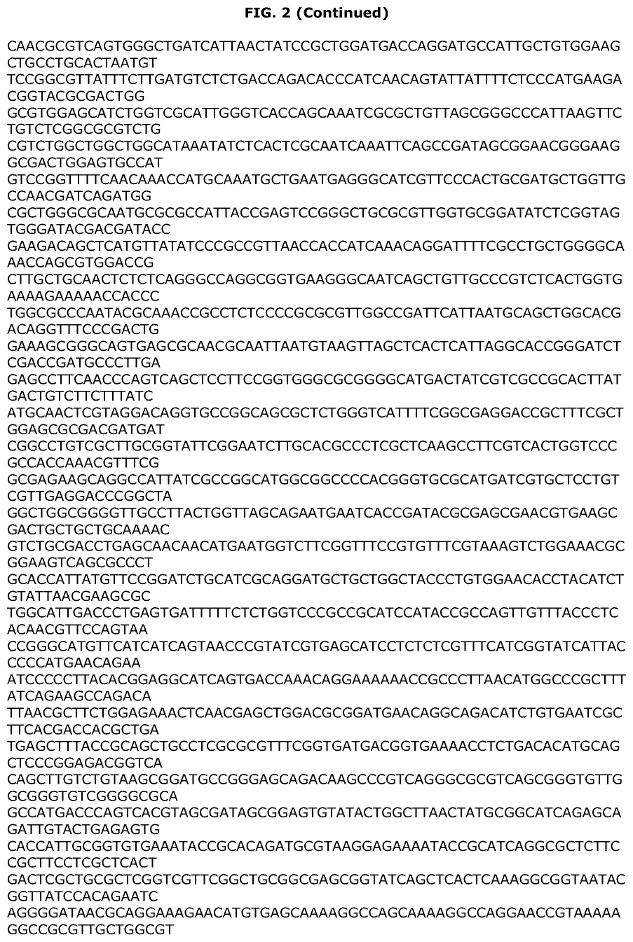

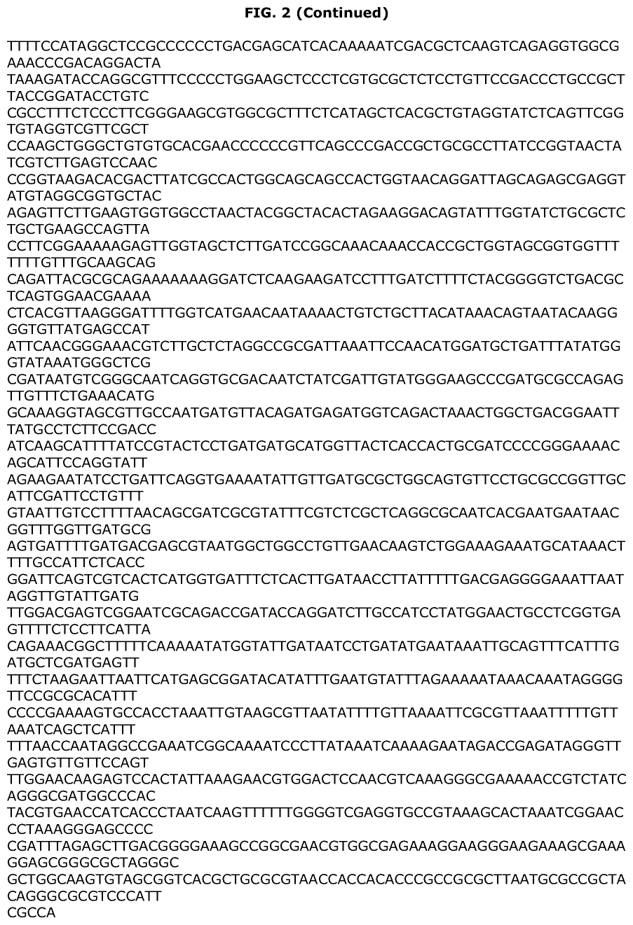

Consensus based design was performed using the alignment sequence of MHETase and its closest relatives. From this, a number of different combinatorial MHETase sequences were constructed using different consensus threshold: 95%, 90%, 85%, 80%, 75%, 70%, 65%, 60%, 55% and 50%. For example, the consensus design at a 95% threshold represents all of the differences that are observed in 95% of the aligned sequences, but not in WT MHETase. The amino acid sequences of the WT MHETase (SEQ ID NO:1) and of the different consensus designs (SEQ ID NOs:2-36) are shown in FIG. 1. The nucleic acid sequences of the WT MHETase and of the different consensus designs (SEQ ID NOs:37-72) are shown in FIG. 2. The different consensus designs resulting from thresholds of 95%, 90%, 85%, 80%, 75%, 70%, 65%, 60%, 55% and 50%, are herein referred to as Round 1 Consensus A, B, C, D, E, F, G, H, I and J, respectively.

The activity/expression of the MHETase was measured using the chromogenic assay as described above using whole cell suspension.

As shown in FIG. 3, the variant “Round 1 Consensus A” (SEQ ID NO:73) showed more whole-cell activity than the WT MHETase or other consensus designs. Round 1 Consensus A contained 2 amino acid substitutions when compared to the WT; namely, N156G and T159V. As an initial study, point mutations were added to this variant based on other consensus residues identified through multiple sequence alignment to identify mutations that further stabilize/improve MHETase activity and/or expression. The remaining variants (mutants; SEQ ID NOs:3-36) comprise one of the following point mutations when compared to the Round 1 Consensus A sequence (the nomenclature refers to the amino acid position of the wild-type MHETases, SEQ ID NO:1):
T68V, A78P, E110A, M117L, E128Q, S131G, N134D, A161G, G156N, D191L, M192Y, S196A, Y197V, G204A, A207L, A216P, E226N, L234A, S235A, P255V, G258A, S260A, T264L, T265L, N284L, L295V, S296A, T355R, A377P, S463L, A493P, Y503W, E594A and N496S

Also shown in FIG. 3, point mutants S196A, Y197V, S235A, P255V, S260A, S286A, Y503W when made onto Round 1 Consensus A sequence, showed much higher whole cell activity when expressed in E. coli.

Soluble cell lysate was run via SDS-PAGE and stained with NTA-Atto550 to identify expression rates of the enzyme. MHETase runs at 64 kDa on the gel are shown in FIG. 4. This gel shows that most of the MHETase mutants (including the controls) are not visible, even under this more specific staining method. This result is consistent with low soluble protein expression. Round 1 Consensus A and variants comprising point mutant S196A, Y197V, S235A, P255V, S260A, S286A or Y503W onto the variant B backbone appeared to show increased heterologous expression in E. coli over wild type (WT).

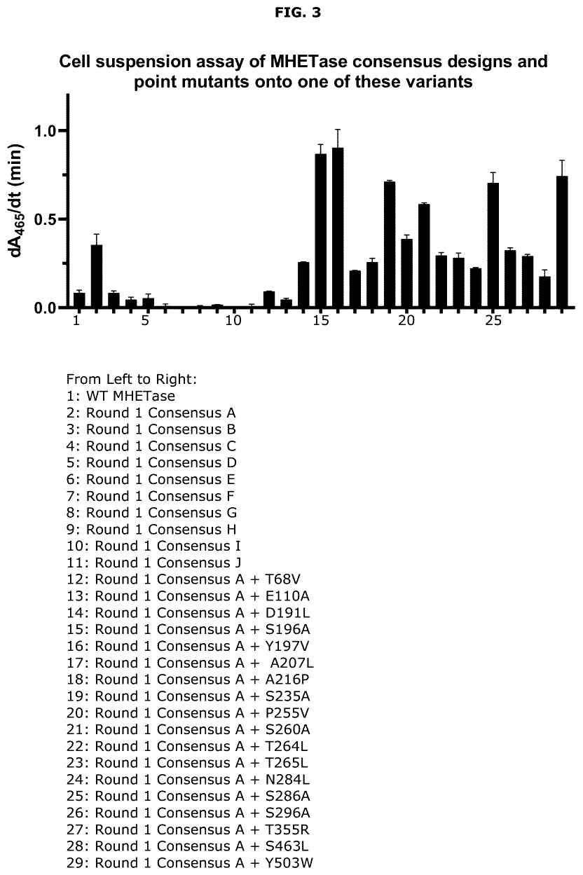

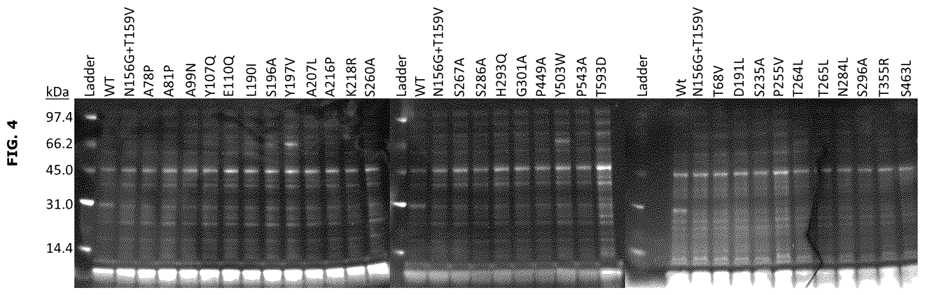

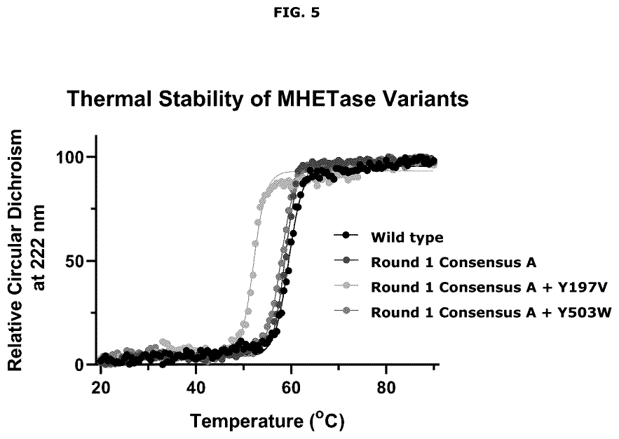

The stability of selected generated variants were also tested using the purified protein variants. As shown in FIG. 5, the thermal melt (Tₘ) of WT MHETase, Round 1 Consensus A and Round 1 Consensus A+Y503W were all approximately 59° C., whereas the introduction of the Y197V point mutation to Round 1 Consensus A reduced the Tₘ to 52° C.

One of the key limitations of the use of wild type MHETase in industrial applications is its low expression in industrial strains. An increase in whole cell activity (a combination of catalytic efficiency and expression yield of active protein), as demonstrated by the variants disclosed herein, advantageously reduces the cost of production and use.

### Example 2: Engineering Mhetases With Improved Activity

To generate variants with improved thermostability, Round 1 Consensus A (containing N156G and T159V point mutations) was selected as a base for further engineering, as Round 1 Consensus A was shown to have improved whole cell MHETase activity (FIG. 3) with little change to thermostability (FIG. 5). In this new engineering process, point mutations were added to Round 1 Consensus A to generate a library of mutants (Round 2) and screened for whole cell activity. The process entails selecting most promising variant and iteratively introducing single point mutations over several design rounds.

In Round 2, the following point mutations where introduced into the Round 1 Consensus A backbone (the nomenclature refers to the amino acid position of the wild-type MHETases, SEQ ID NO:1):
T593D, P543A, Y503W, S463L, P449A, T355R, G301A, S296A, H293Q, S286A, N284L, S267A, T265L, T264L, S260A, P255V, S235A, K218R, A216P, A207L, Y197B, S196A, D191L, L1901, E110A, Y107Q, A99N, A81P, A78P, T68V

The best performing variant selected from Round 2 is “Round 2 Y503W” (containing N156G, T159V and Y503W point mutations; SEQ ID NO: 74).

In Round 3, the following point mutations where introduced into “Round 2 Y503W:
E594A, A493P, A377P, S286A, S267A, T264L, S260A, S196A, M192Y

The best performing variant selected from Round 3 is “Round 3 M192Y” (containing N156G, T159V, M192Y and Y503W point mutations; SEQ ID NO: 75).

In Round 4, the following point mutations where introduced into Round 3 M192Y, wherein ‘+’ denotes the insertion of an amino acid at a particular position:
+N564, S561A, G534A, L486V, A469P, +P467, H467M, W398K, A377P, M361F, I357L, S288T, L282D, S286A, S267A, S260A, Y252F, M233V, G231A, E230H, V2081, Q202P, V200L, T162S, A161S, G156N, V159T, L137V, G130N, R114E, L112G, A79G

The best performing variant selected from Round 4 is “Round 4 G156N” (containing T159V, M192Y and Y503W point mutations; SEQ ID NO: 76).

In Round 5, the following point mutations where introduced into Round 4 G156N, wherein ‘+’ denotes the insertion of an amino acid at a particular position:
N592E, I582V, G562R, R537Q, A494V, W466F, Q461E, M361F, I357L, N316D, I283L, Y252F, V246L, Y242F, G231A, V200L, G164A, I104V, E90A, E71T

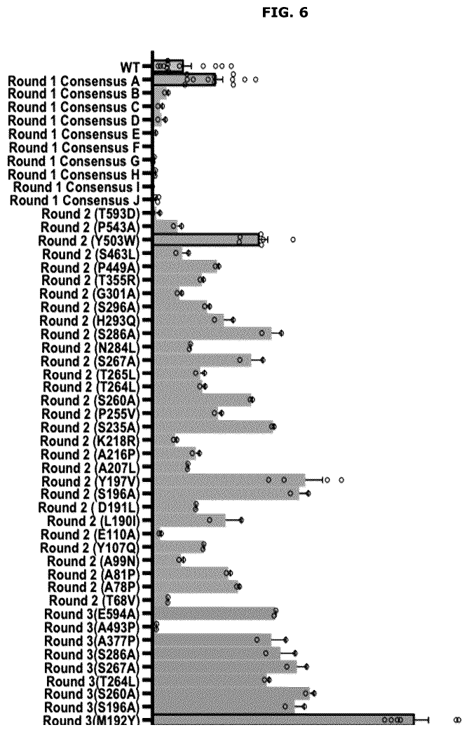

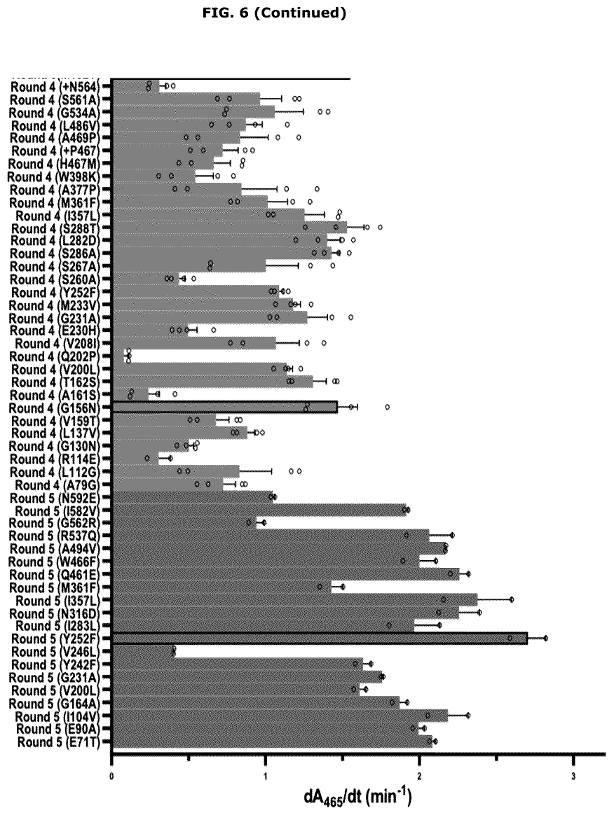

The best performing variant selected from Round 5 is “Round 5 Y252F” (containing T159V, M192Y, Y252F and Y503W; SEQ ID NO: 77), which displayed a 16-fold increase in whole cell activity relative to the wild-type (FIG. 6).

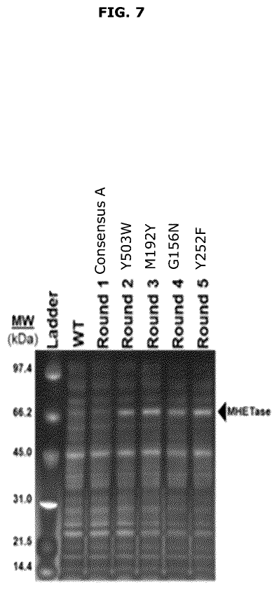

The expression of the best variant from each round was qualitatively measured using an SDS-PAGE stained with the His-tag specific fluorescent label ATTO550. The expression of WT MHETase was too low to be conclusively detected over background endogenous E. coli protein expression. However, by Round 2 of engineering, the variant MHETase (Round 2 Y503W) recombinant expression level was improved to a level of being visibly detectable using ATTO550 fluorescent dye (FIG. 7).

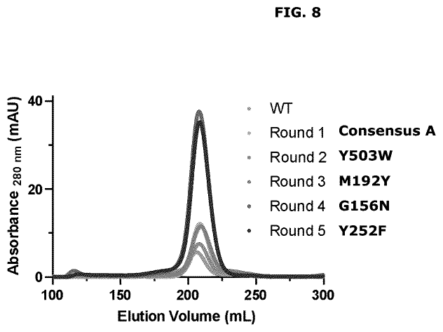

The chromogenic assay described herein was used with purified protein (FIG. 8) to determine the kinetic parameters of these enzymes. The catalytic efficiency (kcat/KM) of all variants remained relatively constant to that of wild-type MHETase. FIG. 9 shows the Michaelis Menten plot of the velocity of enzyme function at various substrate concentrations. From these data, two constants were attained—kcat and KM, as shown in Table 1.

TABLE 1
Michaelis-Menten kinetic parameters
of selected MHETase Variants

| | K_{M} ± SE | k_{cat} ± SE | k_{cat}/K_{M} ± SE |
|---|---|---|---|
| Enzyme Variants | (μM) | (sec⁻¹) | (μM⁻¹ · sec⁻¹) |
| MHETase WT | 21.41 ± 3.63 | 88.69 ± 4.34 | 4.14 ± 0.90 |
| Round 1 Consensus A | 18.55 ± 3.19 | 73.00 ± 3.47 | 3.94 ± 0.86 |
| Round 2 Y503W | 18.07 ± 2.36 | 72.54 ± 2.60 | 4.01 ± 0.67 |
| Round 3 M192Y | 10.24 ± 1.30 | 33.96 ± 0.99 | 3.32 ± 0.52 |
| Round 4 G156N | 11.31 ± 1.81 | 39.59 ± 1.50 | 3.50 ± 0.69 |
| Round 5 Y252F | 9.38 ± 1.69 | 36.13 ± 1.45 | 3.85 0.85 |

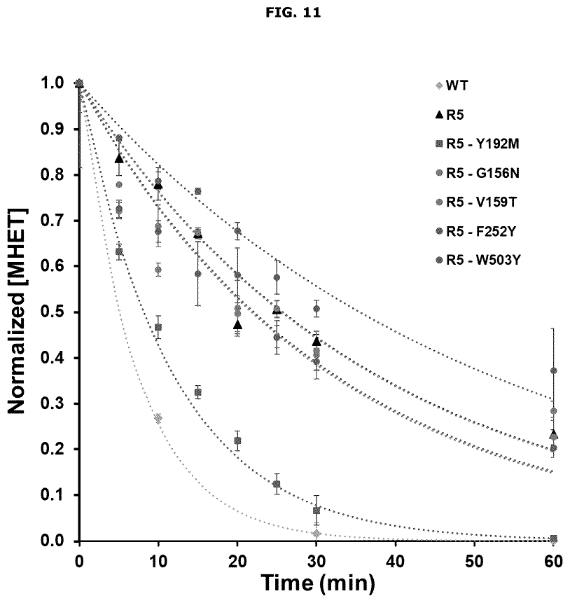

To validate the data observed using the chromogenic assay described herein, the activity of wild-type MHETase and the best variant from Round 5 (Round 5 Y252F; SEQ ID NO: 77) against the natural substrate MHET was determined by high-performance liquid chromatography (HPLC). HPLC was used to measure the concentrations of terephthalic acid (TPA) and MHET in enzyme reactions containing wild-type MHETase or Round 5 Y252F, over time. The data was in agreement with the reductions in k_{cat} and K_{M} observed using the chromogenic assay and confirm that Round 5 Y252F hydrolyses MHET to TPA. (see FIG. 11).

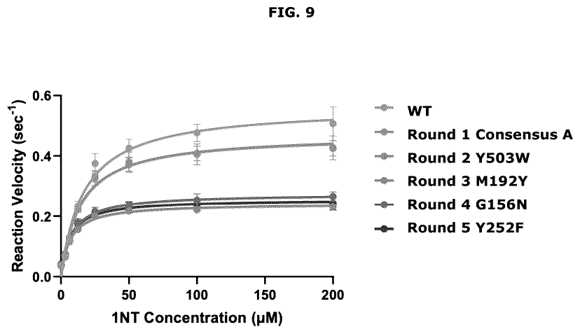

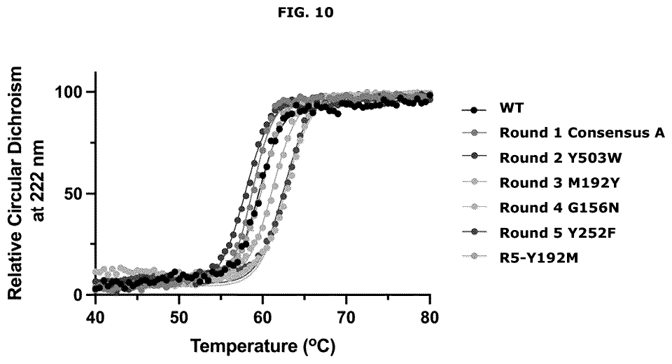

Stability tests on the consensus designs were also performed using the purified protein variants. Mutations at positions 192 and 252 improve the thermal stability of (Tm) relative to WT MHETase (FIG. 10).

Overall, these stabilizing characteristics contribute to the improved expression and activity of Round 5 Y252F relative to wild-type MHETase. Despite these improvements, however, this variant's specific activity is reduced in comparison to the wild type, as demonstrated by its lower k_{cat} (Table 1).

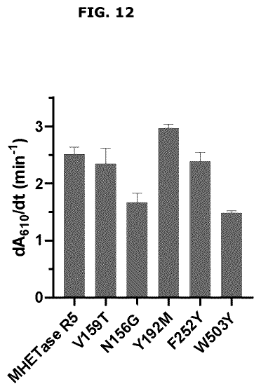

Further engineering was performed to revert some of the individual mutations in Round 5 Y252F to their wild-type residue. In the case of position 156, mutation N156G was reintroduced to the background of MHETase R5 in order to investigate the impact of reverting this residue to the wild-type identity in Round 4. Whole cell activity as determined by FastBlue assay is shown in FIG. 12.

When the mutation M192Y was reverted to the wildtype residue (M) in Round 5 Y252F (“R5-Y192M”; SEQ ID NO: 78), around a 3-fold increase in specific activity was observed relative to Round 5 Y252F (FIG. 11), restoring specific activity to a level that is more comparable to that of the wild-type MHETase of SEQ ID NO:1. The reversions of other point mutations in Round 5 Y252F either reduced or did not significantly alter enzyme activity. The thermal stability of the MHETase variants is shown in Table 2.

TABLE 2
Thermostability of wild-type (WT) MHETase
and selected MHETase Variants

| Enzyme | Tm (° C.) |
|---|---|
| MHETase WT | 59.5 |
| Round 1 Consensus A variant | 59.6 |
| Round 2 Y503W variant | 57.9 |
| Round 3 M192Y variant | 61.2 |
| Round 4 G156N variant | 62.8 |
| Round 5 Y252F variant | 62.6 |
| Round 5-Y192M variant | 59.6 |

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1-48. (canceled)

49. A polypeptide having mono-(2-hydroxyethyl) terephthalate hydrolase (MHETase) activity, wherein the polypeptide comprises an amino acid sequence that (i) has at least 70% sequence identity to SEQ ID NO:1 and (ii) differs from SEQ ID NO:1 by an amino acid substitution at one or more positions selected from the group consisting of
(a) a position that corresponds to amino acid positions 156 of SEQ ID NO:1;
(b) a position that corresponds to amino acid position 159 of SEQ ID NO:1;
(c) a position that corresponds to amino acid position 192 of SEQ ID NO:1; and
(d) a position that corresponds to amino acid position 503 of SEQ ID NO:1,
wherein the MHETase activity of the polypeptide is greater than the MHETase activity of the MHETase of SEQ ID NO: 1.

50. The polypeptide of claim 49, wherein the amino acid sequence of the polypeptide differs from SEQ ID NO:1 by an amino acid substitution at a position that corresponds to amino acid position 156 of SEQ ID NO:1.

51. The polypeptide of claim 49, wherein the amino acid substitution at a position that corresponds to amino acid position 156 of SEQ ID NO:1 is N156G, or a conservative amino acid substitution thereof.

52. The polypeptide of claim 49, wherein amino acid sequence of the polypeptide differs from SEQ ID NO:1 by an amino acid substitution at a position that corresponds to amino acid position 159 of SEQ ID NO:1.

53. The polypeptide of claim 49, wherein the amino acid substitution at a position that corresponds to amino acid position 159 of SEQ ID NO:1 is T159V, or a conservative amino acid substitution thereof.

54. The polypeptide of claim 49, wherein the amino acid sequence of the polypeptide differs from SEQ ID NO:1 by an amino acid substitution at a position that corresponds to amino acid position 252 of SEQ ID NO:1.

55. The polypeptide of claim 49, wherein the amino acid substitution at a position that corresponds to amino acid position 252 of SEQ ID NO:1 is Y252F, or a conservative amino acid substitution thereof.

56. The polypeptide of claim 49, wherein the amino acid sequence of the polypeptide differs from SEQ ID NO:1 by an amino acid substitution at a position that corresponds to amino acid position 503 of SEQ ID NO:1.

57. The polypeptide of claim 49, wherein the amino acid substitution at a position that corresponds to amino acid position 503 of SEQ ID NO:1 is Y503W, or a conservative amino acid substitution thereof.

58. The polypeptide of claim 49, wherein the amino acid sequence of the polypeptide differs from SEQ ID NO:1 by amino acid substitutions at positions that correspond to amino acid positions 159, 252, and 503 of SEQ ID NO:1.

59. The polypeptide of claim 49, wherein the amino acid sequence of the polypeptide differs from SEQ ID NO:1 by amino acid substitutions at positions that correspond to amino acid positions 159, 192, 252, and 503 of SEQ ID NO:1.

60. The polypeptide of claim 49, wherein the amino acid sequence of the polypeptide differs from SEQ ID NO:1 by amino acid substitutions at positions that correspond to amino acid positions 159, 192, and 503 of SEQ ID NO:1.

61. The polypeptide of claim 49, wherein the amino acid sequence of the polypeptide differs from SEQ ID NO:1 by amino acid substitutions at positions that correspond to amino acid positions 156, 159, and 503 of SEQ ID NO:1.

62. The polypeptide of claim 49, wherein the amino acid substitutions are T159V, Y252F, and Y503W, or conservative amino acid substitutions of any of the foregoing.

63. The polypeptide of claim 49, wherein the amino acid substitutions are T159V, M192Y, Y252F, and Y503W, or conservative amino acid substitutions of any of the foregoing.

64. The polypeptide of claim 49, wherein the amino acid substitutions are N156G, M192Y, and Y503W, or conservative amino acid substitutions of any of the foregoing.

65. The polypeptide of claim 49, wherein the amino acid substitutions are N156G, T159V, M192Y, and Y503W, or conservative amino acid substitutions of any of the foregoing.

66. A composition comprising the polypeptide of claim 49.

67. A polynucleotide comprising a nucleic acid sequence encoding the polypeptide of claim 49.

68. A host cell comprising the polynucleotide of claim 67.

69. A method of producing a polypeptide having MHETase activity, the method comprising:
(a) providing the polynucleotide of claim 67;
(b) expressing the nucleic acid sequence in a host cell culture, thereby producing the polypeptide; and
(c) collecting the polypeptide produced in (b) from the host cell culture.

70. A method of hydrolysing a mono-(2-hydroxyethyl) terephthalate, the method comprising exposing the mono-(2-hydroxyethyl) terephthalate to the polypeptide of claim 49, under conditions sufficient to convert the mono-(2-hydroxyethyl) terephthalate to terephthalate and ethylene glycol.

71. A method of degrading a plastic product comprising a polyester, the method comprising exposing the plastic product to the polypeptide of claim 49.

72. The method of claim 71, wherein the polyester is selected from the group consisting of polylactic acid (PLA), polytrimethylene terephthalate (PTT), polybutylene terephthalate (PBT), polyethylene isosorbide terephthalate (PEIT), polyethylene terephthalate (PET) polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate) (PEA), poly(glycolic acid) (PGA), poly(lactic-co-glycolic acid) (PLGA) and combinations of any of the foregoing.

73. The method of claim 72, wherein the polyester is polyethylene terephthalate (PET).

74. The method of claim 73, comprising:
(a) exposing the PET to a polyethylene terephthalate esterase (PETase) under conditions sufficient for the PETase to catalyze the conversion of the PET to generate mono-(2-hydroxyethyl) terephthalate (MHET); and
(b) simultaneously or sequentially, exposing the MHET generated in step (a) to the polypeptide of claim 1 under conditions sufficient for the polypeptide to catalyze the hydrolysis of the MHET to produce terephthalate and ethylene glycol.

75. The method of claim 74, further comprising recovering the terephthalate and/or ethylene glycol produced in step (b).

76. A composition comprising the terephthalate and/or ethylene glycol recovered by the method of claim 75.
